(19) United States
(12) Patent Application Publication
(10) Pub. No.: US 2015/0272436 A1
(43) Pub. Date: Oct. 1, 2015

(54) OPHTHALMOLOGIC APPARATUS

(71) Applicant: Kabushiki Kaisha TOPCON, Tokyo (JP)

(72) Inventor: Takefumi HAYASHI, Tokyo (JP)

(73) Assignee: Kabushiki Kaisha TOPCON, Tokyo (JP)

(21) Appl. No.: 14/644,897

(22) Filed: Mar. 11, 2015

(30) Foreign Application Priority Data
Mar. 25, 2014 (JP) 2014-062736

(51) Int. Cl. A61B 3/107; A61B 3/00; A61B 3/15

(52) U.S. Cl. CPC A61B 3/107; A61B 3/152; A61B 3/0008

(57) ABSTRACT

Provided is an ophthalmologic apparatus capable of determining whether measurement light is properly projected onto an eye to be examined. The apparatus includes an ocular characteristics measurement unit that measures an optical characteristic of the eye by projecting measurement light onto the eye and receiving light reflected by the eye, an observation optical system that acquires an image of an anterior ocular segment, a measurement area projection optical system that projects measurement area index light onto a cornea of the eye from a position defined in accordance with an optical axis of the ocular measurement unit, and a control unit that controls the ocular characteristics measurement unit, the observation optical system, and the measurement area projection optical system. The control unit determines whether the measurement light is properly projected onto the eye based on the acquired image of the anterior ocular segment including the measurement area index light.

### CROSS-REFERENCE TO RELATED APPLICATIONS

The present application is based on and claims priority from Japanese Patent Application Number 2014-062736, filed on Mar. 25, 2014, the disclosure of which is hereby incorporated by reference herein in its entirety.

### BACKGROUND

The present invention relates to an ophthalmologic apparatus for measuring ocular characteristics of an eye to be examined by projecting measurement light onto the eye.

An ophthalmologic apparatus for measuring ocular characteristics of an eye to be examined by projecting measurement light thereon has been taught by, for example Patent Document 1 (Japanese Laid-Open Patent Application No. 2010-148589). The ophthalmologic apparatus of Document 1 is configured to receive reflected measurement light that has been projected onto the eye and reflected by the eye with a light-receiving optical system so as to measure or examine ocular characteristics of the eye to be examined.

### SUMMARY

The ophthalmologic apparatus of Document 1 is, however, not capable of determining whether the measurement light is properly projected onto the eye to be examined. Therefore, the ophthalmologic apparatus of Document 1 may measure or examine the ocular characteristics with the improperly projected measurement light. As a result, the ophthalmologic apparatus of Document 1 may erroneously conclude or determine that intraocular optical system of the eye to be examined has disorder or is in an abnormal condition based on the improper measurement results, or the apparatus may need to repeat the measurement.

To solve the above problems, it is an object of the present invention to provide an ophthalmologic apparatus which is capable of determining whether measurement light is properly projected onto an eye to be examined.

To achieve the above object, an aspect of the present invention provides an ophthalmologic apparatus comprising an ocular characteristics measurement unit that measures an optical characteristic of an eye to be examined by projecting measurement light onto the eye and receiving light reflected by the eye, an observation optical system that acquires an image of an anterior ocular segment of the eye, a measurement area projection optical system that projects measurement area index light corresponding to a measurement area of the ocular characteristics measurement unit onto a cornea of the eye from a position defined in accordance with an optical axis of the ocular characteristics measurement unit, and a control unit that controls the ocular characteristics measurement unit, the observation optical system, and the measurement area projection optical system. The control unit determines whether the measurement light is properly projected onto the eye based on the acquired image of the anterior ocular segment which includes the measurement area index light projected onto the cornea of the eye.

### BRIEF DESCRIPTION OF THE DRAWINGS

FIG. 1 is a view showing a schematic structure of an ophthalmologic apparatus according to an embodiment of the present invention.

FIG. 2 is an explanatory view for explaining an optical arrangement of an intraocular pressure measurement unit of the ophthalmologic apparatus according to the embodiment.

FIG. 3 is an explanatory view for explaining an optical arrangement of the intraocular pressure measurement unit showing from a different direction from that of FIG. 2.

FIG. 4 is an explanatory view for explaining an optical arrangement of an ocular characteristics measurement unit of the ophthalmologic apparatus according to the embodiment.

FIG. 5A is an explanatory view showing an intraocular pressure measurement mode.

FIG. 5B is an explanatory view showing an ocular characteristics measurement mode.

FIG. 6A is an explanatory view for explaining a predetermined height HL with respect to positions of a main body of the apparatus and a forehead pad.

FIG. 6B is an explanatory view for explaining a predetermined front position FL with respect to positions of a main body of the apparatus and a forehead pad.

FIG. 7 is an explanatory view showing an image of an anterior ocular segment (cornea) of an eye to be examined displayed on a display, in a case where a center-ring index image of ring indexes for measuring a corneal shape indicating a measurement area Am is appropriately located within a pupil and the ring indexes are in a proper condition.

FIG. 8 is an explanatory view showing an image of the anterior ocular segment (cornea) of the eye to be examined displayed on the display, in a case where the center-ring index image of the ring indexes for measuring the corneal shape indicating the measurement area Am is appropriately located within the pupil but the anterior ocular segment (cornea) is shaded by eyelashes since the eyelid is not fully open.

FIG. 9 is an explanatory view showing an image of the anterior ocular segment (cornea) of the eye to be examined displayed on the display, in a case where the eyelid is fully open so that the anterior ocular segment (cornea) is not shaded by the eyelashes but the center-ring index image of the ring index for measuring the corneal shape indicating the measurement area Am is not appropriately located within the pupil.

FIG. 10 is a flowchart showing process or method executed by a control unit to determine whether the measurement light is properly projected.

### DETAILED DESCRIPTION OF THE PREFERRED EMBODIMENTS

Hereinafter, an ophthalmologic apparatus according to an embodiment of the present invention will be explained with reference to the drawings.

### Embodiment

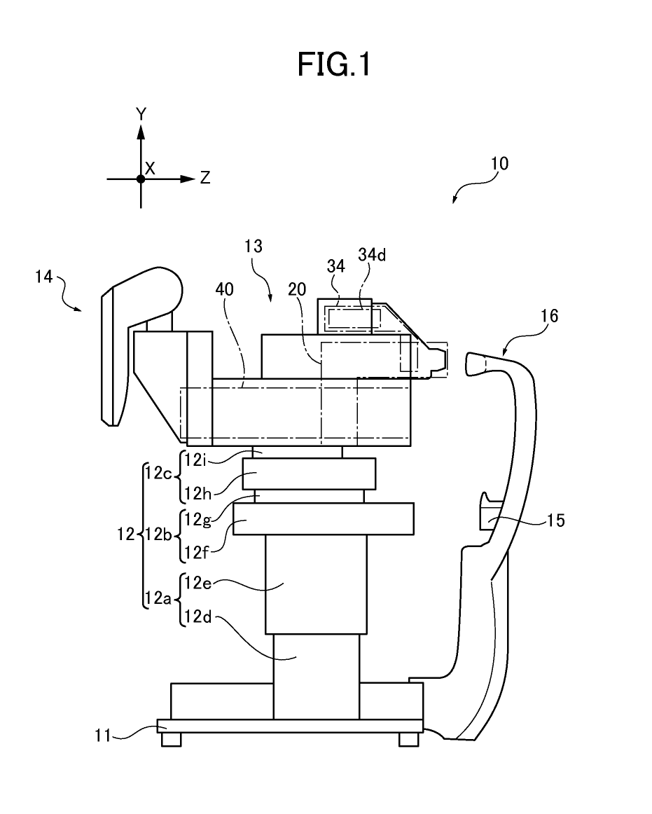

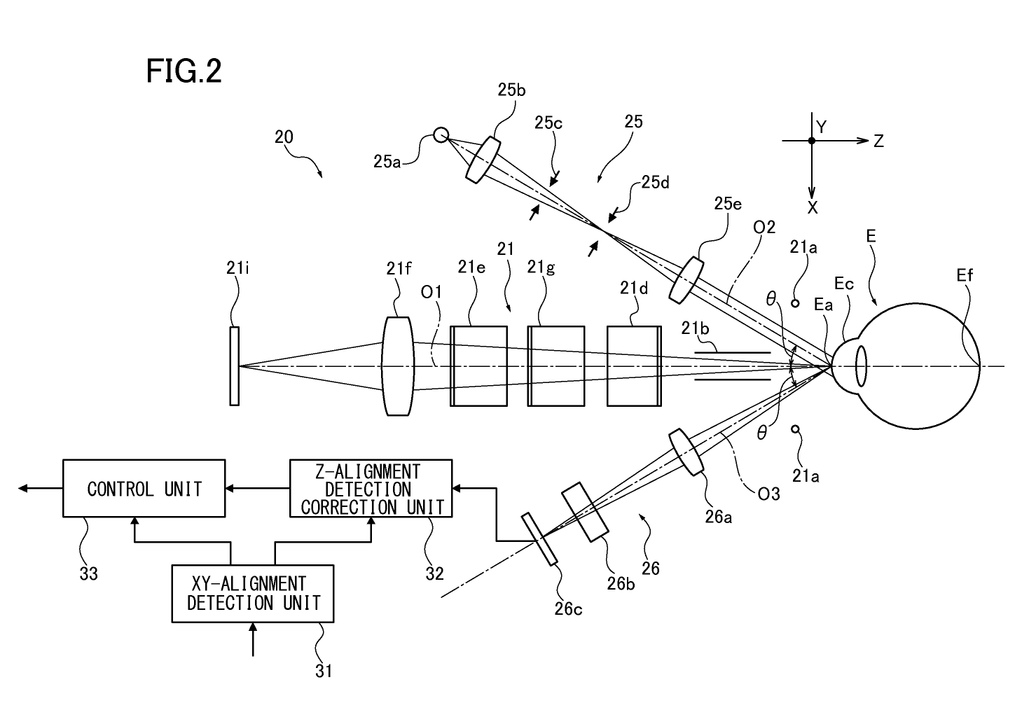

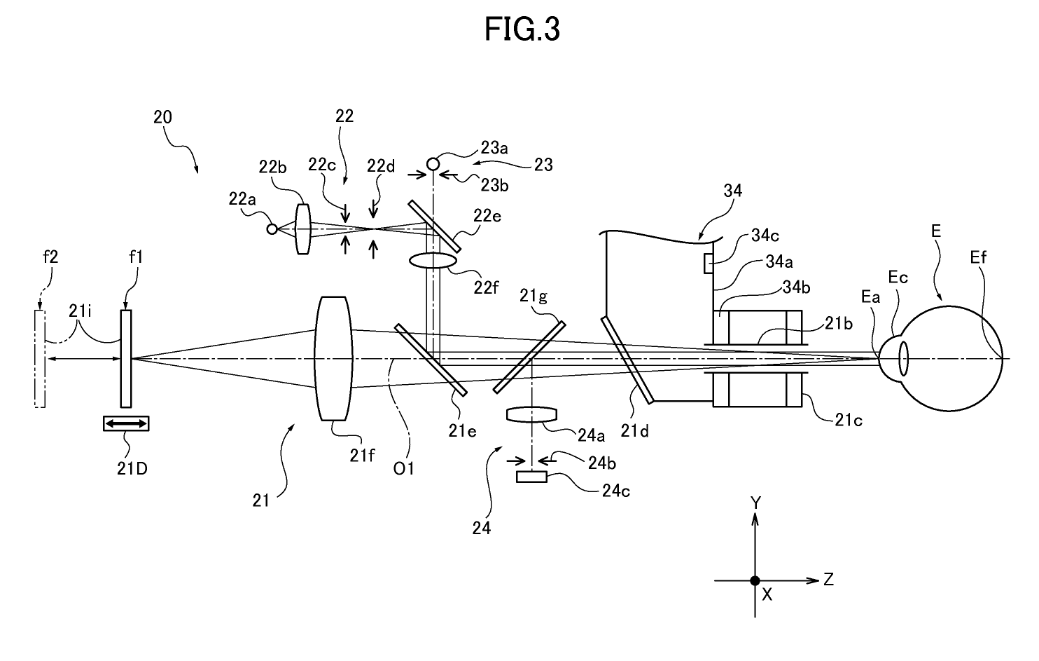

An ophthalmologic apparatus 10 according to an embodiment of the present invention will be explained with reference to FIGS. 1 to 10. As illustrated in FIG. 1, the ophthalmologic apparatus 10 is a composite ophthalmologic apparatus including an intraocular pressure measurement unit 20 adapted to measure intraocular pressure of an eye to be examined E (shown in FIG. 2), and an ocular characteristics measurement unit 40 adapted to measure optical characteristics (ocular characteristics other than the intraocular pressure) of the eye E. FIGS. 2 and 3 schematically show the eye E with its ocular fundus (retina) Ef, cornea (anterior ocular segment) Ec, and corneal apex Ea. The apparatus 10 further includes a base 11 and a main body 13 connected to the base 11 via the driving unit 12. The intraocular pressure measurement unit 20 and the ocular characteristics measurement unit 40 are installed inside of the main body 13, and a display unit (display) 14, a chin rest 15, and a forehead pad 16 are installed outside of the main body 13.

The display 14 is a liquid crystal display and controlled by a control unit 33 (shown in FIG. 2) to display an image of the anterior ocular segment of the eye to be examined E, examination results, and the like. The display 14 of this embodiment is designed as a touch panel such that an examiner can manipulate (operate) the intraocular pressure measurement unit 20 and ocular characteristics measurement unit 40 to carry out the measurements and can move the main body 13 by touching the display 14. The display 14 also displays a switchover icon in which the examiner touches to switchover measurement modes from one to another. It is also possible to provide a measurement start switch in which the examiner manipulates to carry out the measurements. Similarly, it is possible to provide a control lever and a switch to move the main body 13.

The chin rest 15 and forehead pad 16 are firmly installed at the base 11 to fix the face of the subject (patient), i.e., a position of the eye to be examined E, relatively to the main body 13 during the measurements. The chin rest 15 is a place to rest the chin of the subject, and the forehead pad 16 is a place to abut the forehead of the subject. In the ophthalmologic apparatus 10 according to the embodiment, the display 14 is generally provided on the examiner's side, while the chin rest 15 and forehead pad 16 are provided on the subject's side. The display 14 is rotatably connected to the main body 13 and able to turn to the opposite side (i.e., the subject's side), sideways (X-axis direction), and the like. The main body 13 can be moved relatively to the base 11, i.e., relatively to the eye to be examined E (face of the subject) fixed by the chin rest 15 and forehead pad 16 by using the driving unit 12.

The driving unit 12 is adapted to move the main body 13 relatively to the base 11 in the up-and-down direction (Y-axis direction), the forward-and-backward direction (Z-axis direction, i.e., left-and-right direction of FIG. 1), and the left-and-right direction (X-axis direction, i.e., a direction perpendicular to the paper of FIG. 1). Note the upside in the up-and-down direction (Y-axis direction), the subject's side in the forward-and-backward direction (Z-axis direction), and the near side of FIG. 1 in the left-and-right side direction (X-axis direction) are defined as positive sides respectively (see arrows illustrated in FIGS. 1 and 2). In this embodiment, the driving unit 12 includes a Y-axis driving part 12a, a Z-axis driving part 12b, and an X-axis driving part 12c.

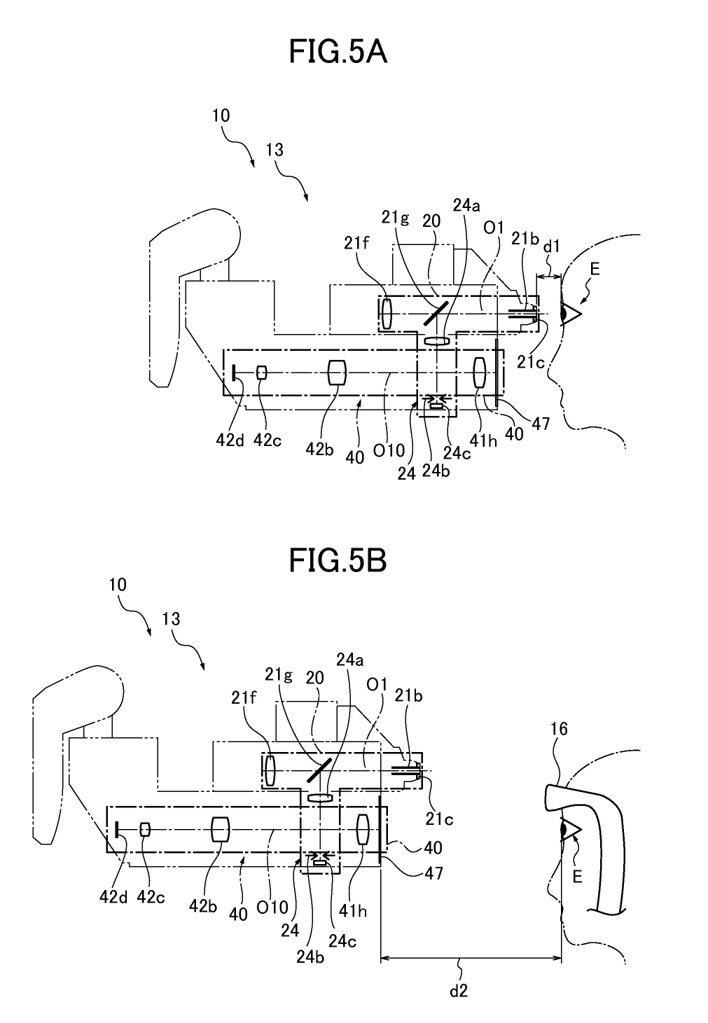

The Y-axis driving part 12a is connected to the base 11 and moves or displaces the main body 13 in the Y-axis direction (up-and-down direction) through the Z-axis driving part 12b and X-axis driving part 12c. The Y-axis driving part 12a has a supporting column 12d and a Y-axis moving frame 12e. The supporting column 12d is fixed to the base 11 and extended from the base 11 in the Y-axis direction. The Y-axis moving frame 12e can cover the supporting column 12d and is attached to the supporting column 12d such that the frame 12e can relatively move in the Y-axis direction through a Y-axis guide member (not shown). The movable range of the Y-axis moving frame 12e relatively to the supporting column 12d (i.e., to the base 11) is set such that the intraocular pressure measurement unit 20 can be moved to the lowermost position in an intraocular pressure measurement mode (shown in FIG. 5A), and the ocular characteristics measurement unit 40 can be moved to the uppermost position in an ocular characteristics measurement mode (shown in FIG. 5B).

Although not illustrated, an elastic member is inserted between the Y-axis moving frame 12e and the supporting column 12d to apply force to push the Y-axis moving frame 12 to the upper side. In this embodiment, the elastic member is a tension spring formed by winding a wire in helical shape, and the Y-axis moving frame 12e is connected to the supporting column 12d via the elastic member. As explained above, the movement of the Y-axis moving frame 12e relatively to the supporting column 12d (i.e., the base 11) is restricted in the Y-axis direction by the Y-axis guide member. Note that the elastic member is not limited to a tension spring. As long as the elastic member is capable of supporting the Y-axis moving frame 12e with the supporting column 12d (i.e., the base 11) by applying force in the Y-axis direction; the elastic member is replaceable with other members such as a compression spring.

The Y-axis driving part 12a includes a driving force transmitting mechanism (not shown) for applying force to move the Y-axis moving frame 12e in the Y-axis direction relatively to the supporting column 12d. Specifically, the Y-axis driving part 12a applies force from the driving force transmitting mechanism to the Y-axis moving frame 12e to move the Y-axis moving frame 12e upward (positive side on the Y-axis) or downward (negative side on the Y-axis) from a balanced position of the frame 12e with the elastic member.

The Z-axis driving part 12b is connected to the Y-axis moving frame 12e (Y-axis driving part 12a) and moves or displaces the main body 13 in the Z-axis direction (forward-and-backward direction) relatively to the Y-axis driving part 12a (i.e., the base 11) through the X-axis driving part 12c. The Z-axis driving part 12b has a Z-axis supporting table 12f and a Z-axis moving table 12g. The Z-axis supporting table 12f is fixed to the Y-axis moving frame 12e and moved in the Y-axis direction together with the Y-axis moving frame 12e. The Z-axis supporting table 12f supports the Z-axis moving table 12g through a Z-axis guide member (not shown) such that the Z-axis moving table 12g can relatively move in the Z-axis direction. The Z-axis driving part 12b includes a driving force transmitting mechanism (not shown) for applying force to move the Z-axis moving table 12g in the Z-axis direction relatively to the Z-axis supporting table 12f. The Z-axis driving part 12b applies force from the driving force transmitting mechanism to the Z-axis moving table 12g to move the Z-axis moving table 12g in the Z-axis direction appropriately.

The X-axis driving part 12c is connected to the Z-axis moving table 12g (Z-axis driving part 12b) and moves or displaces the main body 13 in the X-axis direction (left-and-right direction) relatively to the Z-axis driving part 12b (i.e., the base 11). The X-axis driving part 12c has an X-axis supporting table 12h and an X-axis moving table 12i. The X-axis supporting table 12h is fixed to the Z-axis moving table 12g and moved in the Z-axis direction together with the Z-axis moving table 12g. The X-axis supporting table 12h supports the X-axis moving table 12i through an X-axis guide member (not shown) such that the X-axis moving table 12i can relatively move in the X-axis direction. The X-axis driving part 12c includes a driving force transmitting mechanism (not shown) for applying force to move the X-axis moving table 12i in the X-axis direction relatively to the X-axis supporting table 12h. The X-axis driving part 12c applies force from the driving force transmitting mechanism to the X-axis moving table 12i to move the X-axis moving table 12i in the X-axis direction appropriately. The main body 13 is fixed to the X-axis moving table 12i through a mounting member (not shown) on which the intraocular pressure measurement unit 20 and the ocular characteristics measurement unit 40 are mounted.

Accordingly, the driving unit 12 is adapted to move the main body 13 relatively to the base 11 in the up-and-down direction (Y-axis direction), the forward-and-backward direction (Z-axis direction), and the left-and-right direction (X-axis direction) by driving the Y-axis driving part 12a, Z-axis driving part 12b, and X-axis driving part 12c appropriately. Although it is not shown, the Y-axis driving part 12a, Z-axis driving part 12b, and X-axis driving part 12c are respectively connected to the control unit 33 (shown in FIG. 2), and each driving part is controlled by the control unit 33. The control unit 33 represents an electric control system of the ophthalmologic apparatus 10 and centrally controls each unit of the apparatus 10 in accordance with a program installed in a built-in memory.

To facilitate understanding, a cover member (e.g. a case) to form an external overall appearance is not shown in FIG. 1. The base 11, driving unit 12, and main body 13 are all covered by the cover member. Further, although the Y-axis driving part 12a, the Z-axis driving part 12b, and the X-axis driving part 12c are simply piled up in the Y-axis direction as illustrated in FIG. 1; these parts are not plainly divided into the Y-axis direction but are partially overlapped in view from the perpendicular direction to the Y-axis direction (i.e., the X-axis direction or Z-axis direction). Therefore, it is possible to move the main body 13 relatively to the base 11 appropriately as preventing the height of the driving unit 12 (i.e., the ophthalmologic apparatus 10) from increasing.

The main body 13 is equipped with the intraocular pressure measurement unit 20 and the ocular characteristics measurement unit 40. The intraocular pressure measurement unit 20 is adapted to measure the intraocular pressure of the eye to be examined E. The ocular characteristics measurement unit 40 is adapted to measure optical characteristics (ocular characteristics) of the eye to be examined E. Particularly, the ocular characteristics measurement unit 40 of this embodiment measures a shape of a cornea Ec and an ocular refractive power (spherical power, cylindrical power, cylinder axis angle, and the like) of the eye E. A main optical axis O1 (explained later) of the intraocular pressure measurement unit 20 of the ophthalmologic apparatus 10 according to the embodiment is set above (positive side on the Y-axis direction) with respect to a main optical axis O10 of the ocular characteristics measurement unit 40 (shown in FIGS. 5A and 5B). Therefore, the intraocular pressure measurement unit 20 is generally located above the ocular characteristics measurement unit 40. Note that the intraocular pressure measurement unit 20 and the ocular characteristics measurement unit 40 of the embodiment are not individually separated from each other. The optical system of the intraocular pressure measurement unit 20 and the ocular characteristics measurement unit 40 can optically be crossed with each other, resulting in partially mixing the intraocular pressure measurement unit 20 and the intraocular characteristics measurement unit 40 to form an integral system.

The optical arrangement of the intraocular pressure measurement unit 20 will be explained with reference to FIGS. 2 and 3. The intraocular pressure measurement unit 20 is a non-contact tonometer. As shown in FIGS. 2 and 3, the intraocular pressure measurement unit 20 includes an anterior ocular segment observation optical system 21, an XY-alignment index projection optical system 22, a fixation target projection optical system 23, an applanation detection optical system 24, a Z-alignment index projection optical system 25, and a Z-alignment light detection optical system 26.

The anterior ocular segment observation optical system 21 observes the anterior ocular segment and performs XY-alignment (alignment in a direction along the XY plane) to the eye to be examined E. The anterior ocular segment observation optical system 21 includes illumination light sources for the anterior ocular segment 21a (shown in FIG. 2). The optical system 21 also includes an air-puff nozzle 21b, an anterior ocular segment glass 21c (shown in FIG. 3), a chamber glass 21d, half mirrors 21e and 21g, an objective lens 21f, and a CCD camera 21i on the main optical axis O1. The illumination light sources for the anterior ocular segment 21a are positioned around the anterior ocular segment glass 21c (shown in FIG. 2), and the system 21 has a plurality of the illumination light sources 21a (FIG. 2 illustrates only two of them) to illuminate the anterior ocular segment directly. The air-puff nozzle 21b is adapted to blow air to the eye to be examined E (specifically, to the anterior ocular segment thereof) and is provided with an air compression chamber 34a of an air-puff mechanism 34 (shown in FIG. 3). The CCD camera 21i is adapted to generate an image signal based on an image (of the anterior ocular segment, and the like formed on the light-receiving surface thereof) and output the generated image signal to the control unit 33 (shown if FIG. 2). The control unit 33 displays the image generated by the CCD camera 21 on the display 14 (shown in FIG. 1) and outputs the image to external devices (not shown).

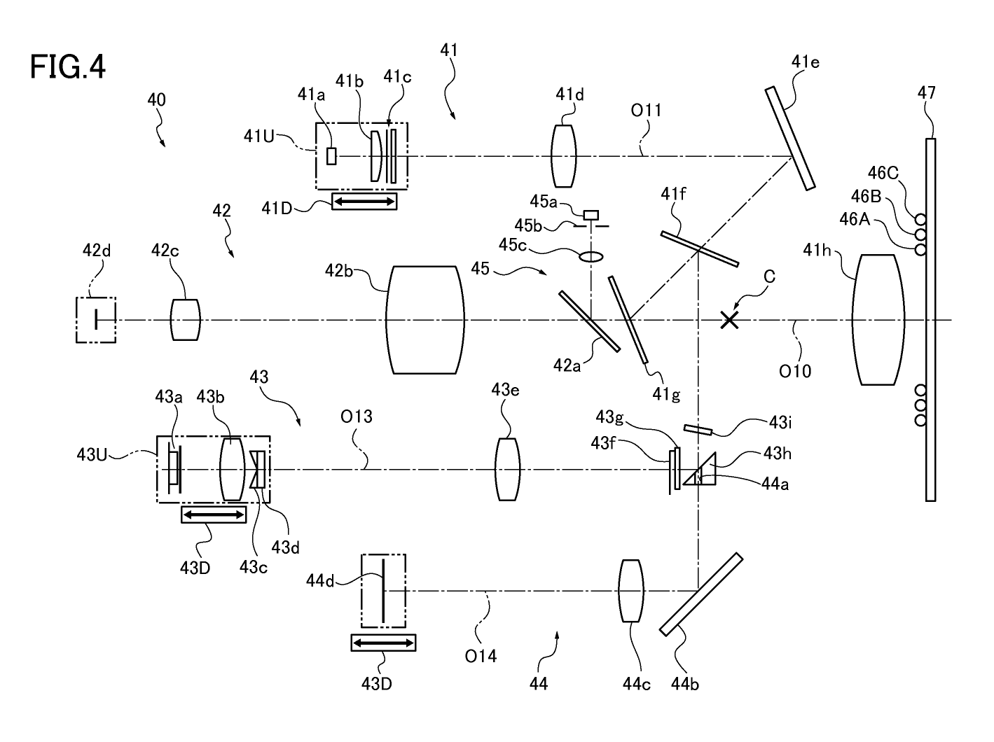

As shown in FIG. 3, the CCD camera 21i is moved along the optical axis O1 by a focusing mechanism 21D. The focusing mechanism 21D is controlled by the control unit 33 to move the CCD camera 21i for focusing on the anterior ocular segment (cornea Ec) of the eye to be examined E. Note the focusing mechanism 21D shares a driving source with a fixation target moving mechanism 41D of a fixation target projection optical system 41 (shown in FIG. 4, explained later). Specifically, the driving source drives both the focusing mechanism 21D in the intraocular pressure measurement mode (shown in FIG. 5A) and fixation target moving mechanism 41D in the ocular characteristics measurement mode (shown in FIG. 5B). The control unit 33 controls the focusing mechanism 21D to move the CCD camera 21i on the optical axis O1 in response to the position of the intraocular pressure measurement unit 20, i.e., the main body 13, to focus on the anterior ocular segment (cornea Ec) of the eye to be examined E.

The control unit 33 according to the embodiment is configured to move or displace the CCD camera 21i from a first focal position f1 to a second focal position f2 on the optical axis O1 by controlling the focusing mechanism 21D. The first focal position f1 is a position where the focus is adjusted to the anterior ocular segment (cornea Ec) of the eye to be examined E when the intraocular pressure measurement unit 20 (the main body 13) is moved to measure the intraocular pressure of the eye E. For example, a distance (interval d1) between the tip of the air-puff nozzle 21b and the eye E is set to 11 mm when the intraocular pressure is measured (shown in FIG. 5A).

The second focal point f2 is a position where the focus is adjusted to the anterior ocular segment (cornea Ec) of the eye to be examined E when the intraocular pressure measurement unit 20 (the main body 13) is sufficiently separated far from the eye E (subject). As explained later, the intraocular pressure measurement unit 20 (the main body 13) is first moved along the Y-axis direction to a height corresponding to the eye to be examined E and then moved along the Z-axis direction to close to the eye E when the measurement mode is switched from the ocular characteristics measurement mode (a measurement mode using the ocular characteristics measurement unit 40) to the intraocular pressure measurement mode (a measurement mode using the intraocular pressure measurement unit 20). This movement is made so that it can avoid the tip of the air-puff nozzle 21b, which is positioned closest to the eye E, being in contact with the eye E accidentally. Also, when the eye to be examined E is switched between the right and left eyes in the intraocular pressure measurement mode, the intraocular pressure measurement unit 20 (the main body 13) is first moved backward (negative side on the Z-axis) from the position where intraocular pressure is measured for one of eyes. The unit 20 is next moved to the other side in the left-and-right direction (X-axis direction) and then aligned to the optical axis of the other eye to be examined E. This movement is made so that it can avoid the tip of the air-puff nozzle 21b being in contact with the subject (specifically, a nose, etc. of the subject).

The distance between the intraocular pressure measurement unit 20 and the eye to be examined E is roughly determined based on the relative position of the main body (the intraocular pressure measurement unit 20) and the base 11. Therefore, the control unit 33 according to the embodiment switches the position of the CCD camera 21i between the first focal position f1 and the second focal position f2 in accordance with the relative position of the main body 13 (i.e., the intraocular pressure measurement unit 20) and the base 11, i.e., the Z-axis position of the driving unit 12 (specifically, the Z-axis driving part 12b). Note that the focusing mechanism 21D of the embodiment shares the driving source with the fixation target moving mechanism 41D of the fixation target projection optical system 41. However, it should not be limited thereto, and is possible to share the driving source with an index moving mechanism 43D of a ring index projection optical system for measuring ocular refractive power 43 and a light-receiving optical system 44 (shown in FIG. 4).

In the anterior ocular observation optical system 21, the illumination light sources for the anterior ocular segment 21a (shown in FIG. 2) illuminates the eye to be examined E (specifically, the anterior ocular segment thereof) and the CCD camera 21i acquires an anterior ocular segment image of the eye E. The image (to be specific, a light flux reflected by the eye E) passes through outside of the air-puff nozzle 21b, the anterior ocular segment glass 21c (including a glass plate 34b as explained later), chamber glass 21d, and half mirrors 21g, 21e. The light flux passed through them is then condensed by the objective lens 21f and forms the anterior ocular segment image on the light-receiving surface of the CCD camera 21i. The anterior ocular segment observation optical system 21 (CCD camera 21i) outputs signals representing the formed anterior ocular segment image to the control unit 33. The control unit 33 displays the anterior ocular segment image acquired by the anterior ocular segment observation optical system 21 (CCD camera 21i) on the display 14 (shown in FIG. 1).

In the anterior ocular observation optical system 21, a reflected light flux that has been projected onto the eye to be examined E as an XY-alignment index light flux from the XY-alignment index projection optical system 22 and reflected by the cornea Ec is also guided to the light-receiving surface of the CCD camera 21i. To be specific, the reflected light flux passes through inside of the air-puff nozzle 21b, the chamber glass 21d, and half mirrors 21g, 21e. The reflected light flux passed through them is then condensed by the objective lens 21f and guided to the CCD camera 21i. Here, a bright point image is formed on the light-receiving surface of the CCD camera 21i at a position corresponding to the relative position of the main body 13 (intraocular pressure measurement unit 20) and the cornea Ec in the XY direction. The anterior ocular segment observation optical system 21 (CCD camera 21i) then outputs signals representing the formed bright point image to the control unit 33. The bright point image of the XY alignment index light flux corresponds to an image formed on the cornea Ec of the eye E. Hence, the control unit 33 acquires an image (data) of the anterior ocular segment (cornea Ec) including the bright point image and is therefore able to display the image of the anterior ocular segment (cornea Ec) with the bright point image on the display 14. An alignment mark generated by an image generating unit (not shown) is can also be displayed on the display 14.

The XY-alignment index projection optical system 22 projects the index light flux to the cornea Ec of the eye to be examined E from the front thereof. The index light flux functions to adjust the relative position of the eye to be examined E (precisely, the anterior ocular segment (i.e., cornea Ec) thereof) and the intraocular pressure measurement unit 20 in the direction along the XY plane (hereinafter called “XY direction”). In other words, the index light flux is used for performing XY-alignment. Further, the index light flux is also used to detect or measure a deformation amount (degree of applanation) of the cornea Ec of the eye E. The XY alignment index projection optical system 22 includes an XY alignment light source 22a, a condenser lens 22b, an aperture stop 22c, a pinhole plate 22d, a dichroic mirror 22e, and a projection lens 22f, and shares the half mirror 21e with the anterior ocular segment observation optical system 21. The XY alignment light source 22a emits infrared light. The projection lens 22f is disposed on the optical path of the XY alignment index projection optical system 22 such that the emitted light is focused at the pinhole plate 22d. In the XY alignment index projection optical system 22, an infrared light emitted from the XY alignment light source 22a is condensed by the condenser lens 22b, passes through the aperture stop 22c, and is guided to the hole of the pinhole 22d. The light flux passed through the pinhole 22d is reflected by the dichroic mirror 22e to guide to the projection lens 22f. The infrared light flux guided to the projection lens 22f is then collimated into a parallel beam and further guided to the half mirror 21e. The parallel beam is then reflected by the half mirror 21e to be on the optical axis O1 of the anterior ocular segment observation optical system 21. The parallel beam passes through the half mirror 21g and chamber glass 21d and is guided to the inside of the air-puff nozzle 21b to reach the eye to be examined E as the XY alignment index light flux. Although not illustrated, the XY alignment index light flux is reflected by the surface of the cornea Ec so as to form a bright point image between the corneal apex Ea of the cornea Ec and the curvature center of the cornea Ec. Note that the aperture stop 22c is disposed at an optically conjugate position with the corneal apex Ea of the cornea Ec with respect to the projection lens 22f.

The fixation target projection optical system 23 projects a fixation target on the eye to be examined E. The fixation target projection optical system 23 includes a fixation target light source 23a and pinhole plate 23b, and shares the dichroic mirror 22e and the projection lens 22f with the XY alignment index projection optical system 22. The system 23 further shares the half mirror 21e with the anterior ocular segment observation optical system 21. The fixation target light source 23a emits visible light. In the fixation target projection optical system 23, a fixation target light emitted from the fixation target light source 23a is guided to the hole of the pinhole plate 23b and passes through the pinhole plate 23b and the dichroic mirror 22e to be guided to the projection lens 22f. The fixation target light flux is then collimated by the projection lens 22f into a substantially parallel beam and is guided to the half mirror 21e. The parallel beam is reflected by the half mirror 21e to be on the optical axis O1 of the anterior ocular segment observation optical system 21. The parallel beam passes through the half mirror 21g and chamber glass 21d and is guided to the inside of the air-puff nozzle 21b to be projected onto the eye E. The fixation target projection optical system 23 fixes the sight line of the subject (patient) by making the subject gaze the fixation target projected onto the eye E.

As illustrated in FIG. 3, the applanation detection optical system 24 receives a reflected light flux that has been projected onto the eye to be examined E as an XY-alignment index light flux from the XY-alignment index projection optical system 22 and reflected by the cornea Ec and detects a deformation amount (degree of applanation) on the surface of the cornea Ec. The applanation detection optical system 24 includes a lens 24a, pinhole plate 24b and sensor 24c, and shares the half mirror 21g with the anterior ocular observation optical system 21. The lens 24a condenses the reflected light flux, which has been projected as the XY-alignment index light flux and reflected by the cornea Ec, to the hole of the pinhole plate 24b when the surface of the cornea Ec is flat. The pinhole plate 24b is disposed such that the condensed light flux passes through the hole of the pinhole plate 24b. The sensor 24c is a light-receiving sensor in which outputs a signal proportional to the received light intensity. In this embodiment, the sensor 24 c consists of a photodiode. The applanation detection optical system 24 (the sensor 24c) outputs the signal proportional to the received light intensity to the control unit 33.

As explained above, the reflected light flux that has been projected as the XY-alignment index light flux and reflected by the surface of the cornea Ec (corneal surface) of the eye to be examined E passes through the inside of the air-puff nozzle 21b and the chamber glass 21d and is guided to the half mirror 21g. In the applanation detection optical system 24, a part of the reflected light flux is reflected by the half mirror 21g and is guided to the lens 24a. The reflected light flux is then condensed by the lens 24a and is guided to the pinhole plate 24b. Here, the air-puff mechanism 34 (shown in FIG. 1; explained later) blows air from the air-puff nozzle 21b to the eye E such that the surface of the corner Ec is gradually deformed to be flat. As a result, the light intensity received by the sensor 24c increases since the lens 24a is configured to condense the whole reflected light flux to pass the hole of the pinhole plate 24b when the surface of the cornea Ec is flat. In other words, when the surface of the cornea Ec is not adequately flat, the lens 24a is not able to condense the reflected light flux sufficiently, resulting in only partial light flux passes through the pinhole plate 24b to reach the sensor 24c. Therefore, the applanation detection optical system 24 can detect or determine that the surface of the cornea Ec becomes flat (applanation) by detecting the moment when the received light intensity is the greatest. Consequently, the applanation detection optical system 24 can detect the surface shape (applanation) of the cornea Ec deformed by the air blown from the air-puff nozzle 21b, and the sensor 24c functions as a light-receiving unit for the detection, which receives the light reflected by the cornea Ec.

As shown in FIG. 2, the Z-alignment index projection optical system 25 obliquely projects a Z-alignment index light flux (an index parallel beam for Z-axis alignment) onto the cornea Ec of the eye to be examined E. The Z-alignment index projection optical system 25 includes a Z-alignment light source 25a, a condenser lens 25b, an aperture stop 25c, a pinhole plate 25d, and a projection lens 25e on an optical axis O2. The Z-alignment light source 25a emits an infrared light (for example, a wavelength of 860 nm). The aperture stop 25c is disposed at an optically conjugate position with the corneal apex Ea of the cornea Ec with respect to the projection lens 25e. The projection lens 25e is disposed such that the focal point of the projection lens 25e is coincided with the hole of the pinhole plate 25d. In the Z-alignment index projection optical system 25, the infrared light flux emitted from the Z-alignment light source 25a is condensed by the condenser lens 25b to pass through the aperture stop 25c and is guided to the pinhole plate 25d. The light flux passed through the pinhole plate 25d is guided to the projection lens 25e and is collimated into a parallel beam. The parallel beam is then guided to the cornea Ec. The infrared light flux guided to the cornea Ec (Z-alignment index light flux) is formed into a bright point image inside the eye to be examined E and is reflected by the surface of the cornea Ec.

The Z-alignment detection optical system 26 receives the reflected light that has been projected as a Z-alignment index light flux and reflected by the cornea Ec from a direction symmetric to the optical axis O2 with the optical axis O1.

The Z-alignment detection optical system 26 detects a positional relationship of the main body (the intraocular pressure measurement unit 20) and the cornea Ec in the Z-axis direction. The Z-alignment detection optical system 26 includes an imaging lens 26a, a cylindrical lens 26b, and a sensor 26c on an optical axis O3. The cylindrical lens 26b has positive power in the Y-axis direction. The sensor 26c is a light-receiving sensor to detect a light-receiving point on the light-receiving surface and is configured with a line sensor or a position sensitive detector (PSD). In this embodiment, the sensor 26c is configured with a line sensor and connected to a Z-alignment detection correction unit 32.

In the Z-alignment detection optical system 26, the reflected light flux that has been projected by the Z-alignment index projection optical system 25 and reflected by the cornea Ec, is guided to the imaging lens 26a. The reflected light flux is condensed by the imaging lens 26a and guided to the cylindrical lens 26b. The condensed light flux is then further condensed in the Y-axis direction by the cylindrical lens 26b and is formed into a bright point image on the sensor 26c. The sensor 26c is disposed at an optically conjugate position with the bright point image formed inside the eye to be examined E with respect to the imaging lens 26a on the X-Z plane. Further, the sensor 26c is optically conjugated with the corneal apex Ea with respect to the imaging lens 26a and cylindrical lens 26b on the Y-Z plane. Accordingly the sensor 26c optically has a conjugate relationship with the aperture stop 25c. Note that the magnification is set such that the image of the aperture stop 25c becomes smaller than the detection range of the sensor 26c. This allows the reflected light flux on the surface of the cornea Ec to enter the sensor 26c efficiently even if the position of the cornea Ec is slightly moved in the Y-axis direction. The Z-alignment detection optical system 26 (the sensor 26c) outputs signals representing the bright point image formed on the sensor 26c to the Z-alignment detection correcting unit 32.

As shown in FIG. 3, the air-puff mechanism 34 is equipped with the air compression chamber 34a and an air compression driver 34b (shown in FIG. 1). The air compression driver 34b has a piston movable inside the air compression chamber 34a and is disposed above the intraocular pressure measurement unit 20 in the main body 13. The air compression driver 34b is driven or controlled by the control unit 33 to compress air in the air compression chamber 34a. The air-puff nozzle 21b is attached to the air compression chamber 34a with a transparent glass plate 34b on a side of the chamber 34a, and the chamber glass 21d is attached thereto on the other side. Therefore, the air compression chamber 34a does not disturb the abovementioned function of the anterior ocular observation optical system 21. The air compression chamber 34a includes a pressure sensor 34c to detect air pressure in the air compression chamber 34a. Although not shown, the pressure sensor 34c is connected to the control unit 33 and outputs signals in response to the detected air pressure to the control unit 33. The air compression driver 34d is controlled by the control unit 33 to compress the air in the air compression chamber 34a and blows the air to the cornea Ec of the eye to be examined E from the air-puff nozzle 21b. Further, the pressure sensor 34c can detect air pressure in the air compression chamber 34a, i.e., pressure of the air blown to the cornea Ec from the air-puff nozzle 21b. Note that it is possible to change the air pressure against time of the air blown from the air-puff nozzle 21b in accordance with predetermined characteristics instead of having a pressure sensor 34c.

The intraocular pressure measurement unit 20 includes a driving mechanism which is connected to the control unit 33 and adapted to control turning on/off the illumination light sources for the anterior ocular segment 21a, the XY-alignment light source 22a, the fixation target light source 23a, and the Z-alignment light source 25a. The intraocular pressure measurement unit 20 is controlled by the control unit 33 to turn on/off the illumination light sources for the anterior ocular segment 21a, the XY-alignment light source 22a, the fixation target light source 23a, and the Z-alignment light source 25a using the driving mechanism. As explained above, the intraocular pressure measurement unit 20 is controlled by the control unit 33 to move the CCD camera 21i on the optical axis O1 using the focusing mechanism 21D. The intraocular pressure measurement unit 20 is also controlled by the control unit 33 to form an image in accordance with the image signals outputted from the CCD camera 21i and to display the formed image on the display 14.

Next, overall operation for measuring an intraocular pressure of the eye to be examined E using the intraocular pressure measurement unit 20 will be explained. The operation explained below is executed by the control unit 33.

First, the examiner turns on a power switch of the ophthalmologic apparatus 10 and manipulates the display (touch panel) to measure the intraocular pressure using the intraocular pressure measurement unit 20. The intraocular pressure measurement unit 20 then moves its positions to be the intraocular pressure measurement mode (as illustrated in FIG. 5A) and turns on the illumination light sources for the anterior ocular segment 21a, the XY-alignment light source 22a, the fixation target light source 23a, and Z-alignment light source 25a. Note that the intraocular pressure measurement unit 20 may periodically turn on/off the light sources 21a, 22a, 23a, and 25a with different cycles, whereby it becomes possible to distinguish the light sources.

As shown in FIG. 3, the intraocular pressure measurement unit 20 turns on the fixation target light source 23a of the fixation target projection optical system 23 to project a fixation target onto the eye to be examined E such that the sight line of the subject is fixed thereto. The intraocular pressure measurement unit 20 turns on the XY-alignment light source 22a of the XY-alignment index projection optical system 22 to project a parallel beam onto the cornea Ec. The parallel beam is reflected on the cornea Ec and is received by the CCD camera 21i of the anterior ocular segment observation optical system 21 and the sensor 24c of the applanation detection optical system 24. As shown in FIG. 2, the intraocular pressure measurement unit 20 turns on the Z-alignment light source 25a of the Z-alignment index projection optical system 25 to project a parallel beam for Z-alignment onto the cornea Ec. The parallel beam for Z-alignment is reflected on the cornea Ec and is received by the sensor 26c of the Z-alignment light detection optical system 26.

The intraocular pressure measurement unit 20 turns on the illumination light sources for the anterior ocular segment 21a of the anterior ocular observation optical system 21 to illuminate the anterior ocular segment of the eye to be examined E, thereby generating an image of the anterior ocular segment of the eye E on the CCD camera 21i. The intraocular pressure measurement unit 20 then displays the generated image of the anterior ocular segment of the eye E, a bright point image of the XY-alignment index light, and an alignment mark. The examiner manipulates an input section of the display 14 to perform a rough alignment such that the bright point image is displayed on the display 14 by moving the main body 13 in the up-and-down direction and the left-and-right direction. Further, the Z-alignment detection correction unit 32 of the intraocular pressure measurement unit 20 calculates positions of the main body 13 and cornea Ec in the Z-axis direction based on the signals from the sensor 26c of the Z-alignment detection optical system 26 and calculation results of the XY-alignment detection unit 31. The intraocular pressure measurement unit 20 is controlled by the control unit 33 to drive the driving unit 12 (i.e., the Y-axis driving unit 12a, Z-axis driving unit 12b, and X-axis driving unit 12c) in accordance with positions of the main body 13 in the XY direction obtained by the anterior ocular segment observation unit 21 and the calculation results outputted from the Z-alignment detection correction unit 32. With this, the intraocular pressure measurement unit 20 can move the main body 13 in X, Y, Z-directions to perform automatic alignment, in which the main body 13 is properly positioned relatively to the base 11.

After performing the automatic alignment, the control unit 33 controls the air-puff mechanism 34 to blow air from the air-puff nozzle 21b to the cornea Ec of the eye to be examined E. As a result, the surface of the cornea Ec of the eye E is gradually deformed to be flat (applanation). As explained above, the received light intensity detected by the sensor 24c of the applanation detection optical system 24 becomes the greatest when the surface of the cornea Ec becomes flat (applanation). Therefore, the control unit 33 can determine that the surface of the cornea Ec becomes flat based on the change of the received light intensity detected by the sensor 24c. That is to say, the applanation detection optical system 24 (the sensor 24c) can detect the applanation of the cornea Ec. The control unit 33 then calculates the intraocular pressure of the eye E based on the output from the pressure sensor 34c (i.e., the pressure of the air blown to the cornea Ec) and displays the calculated result on the display 14. Note that the control unit 33 may calculate the intraocular pressure of the eye E based on a time period between a start time when the control unit 33 begins blowing the air to the cornea Ec from the air-puff nozzle 21b and a time when the control unit 33 detects or determines that the surface of the cornea Ec becomes flat (applanation).

Next, an optical arrangement of the ocular characteristics measurement unit 40 will be explained with reference to FIG. 4. The ocular characteristics measurement unit 40 measures the corneal shape of the eye to be examined E and the ocular reflective power (spherical power, cylindrical power, and cylinder axis angle) of the eye E.

As shown in FIG. 4, the ocular characteristics measurement unit 40 includes the fixation target projection optical system 41, an observation optical system 42, a ring index projection optical system for measuring ocular refractive power 43, the light-receiving optical system 44, an alignment-light projection system 45, and ring index light sources for measuring the corneal shape 46A, 46B, and 46C. The fixation target projection optical system 41 projects a fixation target onto the ocular fundus Ef of the eye to be examined E to fix and/or fog the eye E. The observation optical system 42 observes the anterior ocular segment (cornea Ec). The ring index projection optical system 43 projects a patterned light flux as the ring index for measuring the ocular reflective power onto the ocular fundus Ef to measure the ocular reflective power. The light-receiving optical system 44 receives an image of the ring index (i.e., patterned light flux) that has been projected onto and reflected by the ocular fundus Ef of the eye E by an imaging element 44d (explained later). As explained later, the ring index projection optical system 43 and the light-receiving optical system 44 configures an ocular refractive power measurement optical system. The alignment-light projection system 45 projects index light flux onto the eye E to detect the alignment in the XY direction. The ring index light sources for measuring the corneal shape 46A, 46B, and 46C project ring index light fluxes onto the cornea Ec of the eye E to measure the shape of cornea Ec. As explained later, the ring index light sources for measuring the corneal shape 46A, 46B, and 46C and the observation optical system 42 configure a corneal shape measurement optical system. The ocular characteristics measurement unit 40 further includes an operation distance detection optical system (not shown) to detect an operation distance between the eye E and the main body 13.

The fixation target projection optical system 41 includes a fixation target light source 41a, a collimator lens 41b, an index plate 41c, a relay lens 41d, a mirror 41e, dichroic mirrors 41f and 41g, and an objective lens 41h on an optical axis O11. The index plate 41c has a target to fix and/or fog the eye to be examined E. The fixation target light source 41a, the collimator lens 41b, and the index plate 41c configure a fixation target unit 41U, and are integrally moved by the fixation target moving mechanism 41D along the optical axis O11 of the fixation target projection optical system 41 so as to fix and/or fog the eye E. Note that the dichroic mirror 41g and the objective lens 41h are positioned on the main optical axis O10 of the ocular characteristics measurement unit 40 (explained later).

The fixation target projection optical system 41 emits visible light from the fixation target light source 41a. The visible light is then collimated by the collimator lens 41b into a parallel beam and passes through the index plate 41c to be a target light flux. The target light flux then passes through the relay lens 41d and is reflected by the mirror 41e. The reflected light flux passes through the dichroic mirror 41f and is guided to the dichroic mirror 41g. In the fixation target projection optical system, the target light flux is then reflected by the mirror 41g toward the main optical axis O10 of the ocular characteristics measurement unit 40 and passes through the objective lens 41h to reach the eye to be examined E. The fixation target projection optical system 41 makes the subject gaze the target light flux (fixation target) projected onto the eye E, thereby fixing the sight line of the subject. Further, the fixation target projection optical system 41 moves the fixation target unit 41U integrally to a position where the target image becomes out of focus, thereby fogging the eye to be examined E.

The observation optical system 42 includes an illumination light source (not shown), a half mirror 42a, a relay lens 42b, an imaging lens 42c, and an imaging element 42d. The observation optical system 42 shares the objective lens 41h and dichroic mirror 41g with the fixation target projection optical system 41. The imaging element 42d is a two-dimensional solid state imaging device, particularly is a CMOS imaging sensor in this embodiment.

The observation optical system 42 emits an illumination light flux from the illumination light source to illuminate the anterior ocular segment (cornea Ec) of the eye to be examined E. The illumination light flux is reflected on the anterior ocular segment and reaches to the objective lens 41h. The reflected illumination light flux then passes through the dichroic mirror 41g and the half mirror 42a. The light flux passed through the mirrors 41g, 42a then reaches to the imaging lens 42c through the relay lens 42b and is formed into an image on the light-receiving surface of the imaging element 42d. The imaging element 42d outputs image signals based on the image to the control unit 33. The control unit 33 displays an image of the anterior ocular segment (cornea Ec) on the display 14 in accordance with the inputted image signals. As explained, the observation optical system 42 can form an image of the anterior ocular segment (cornea Ec) on the imaging element 42d (specifically, on the light-receiving surface thereof) and displays the image of the anterior ocular segment on the display 14. Note that the illumination light source of the observation optical system 42 may be turned off when an ocular refractive power measurement is carried out after the alignment has been performed.

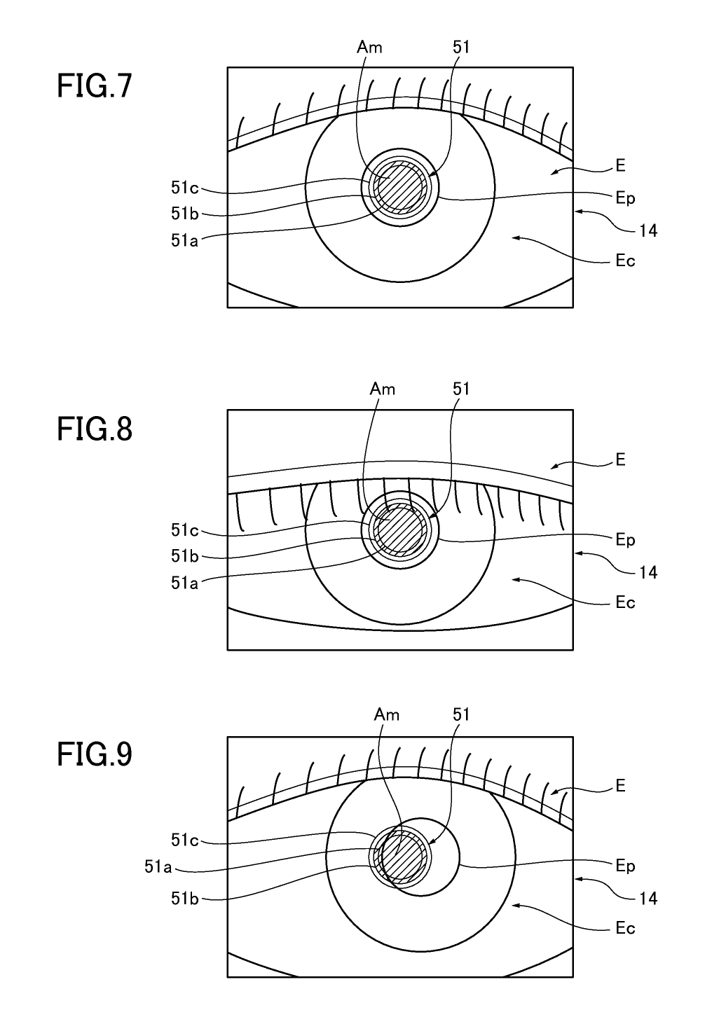

The ring index projection optical system for measuring the ocular refractive power 43 includes a light source for measuring ocular refractive power 43a, a lens 43b, a conical prism 43c, a ring index plate 43d, a lens 43e, a band-pass filter 43f, an aperture ring 43g, a holed prism 43h, and a rotary prism 43i. The ring index projection optical system 43 shares the dichroic mirrors 41f, 41g and the objective lens 41h with the fixation target projection optical system 41. The light source for measuring ocular refractive power 43a and the aperture ring 43g are located in optically conjugate positions. The ring index plate 43d and the ocular fundus Ef of the eye to be examined E are located in optically conjugate positions. Further, the aperture ring 43g is disposed at an optically conjugate position with the pupil Ep of the eye to be examined E via the objective lens 41h (shown in FIG. 7). The light source for measuring ocular refractive power 43a, the lens 43b, the conical prism 43c, and the ring index plate 43d configure the index unit 43U. The index unit 43U is integrally moved by an index moving mechanism 43D along an optical axis O13 of the ring index projection optical system for measuring the ocular refractive power 43. Note the aperture ring 43g is an optical element that has a ring-shaped slit thereon.

The ring index projection optical system for measuring the ocular refractive power 43 emits a light flux from the light source for measuring the ocular refractive power 43a. The emitted light flux is collimated by the lens 43b into a parallel beam and passes through the conical prism 43c. The parallel beam is then passes through a ring index plate 43d to be a patterned light flux which represents the ring index for measuring the ocular refractive power. The patterned light flux passes through the lens 43e, the band-pass filter 43f, and the aperture ring 43g to reach the holed prism 43h. The patterned light flux is then reflected by the reflective surface of the holed prism 43h toward the dichroic mirror 41f through the rotary prism 43i. The reflected patterned light flux is then reflected by the dichroic mirrors 41f and 41g toward the main optical axis O10 of the ocular characteristics measurement unit 40, thereby being formed into the patterned light flux on the ocular fundus Ef of the eye to be examined E by the objective lens 41h.

The light-receiving optical system 44 includes a hole part 44a of the holed prism 43h, a mirror 44b, a lens 44c, and an imaging element 44d. The light-receiving optical system 44 shares the objective lens 41h, dichroic mirrors 41g, 41f with the fixation target projection optical system 41 and shares the rotary prism 43i with the ring index projection optical system 43. The imaging element 44d is a two-dimensional solid state imaging device, particularly is a CCD imaging sensor in this embodiment. The imaging element 44d is moved along an optical axis O14 of the light-receiving optical system 44 by the index moving mechanism 43D of the ring index projection optical system 43 together with the index unit 43U of the ring index projection optical system 43.

The reflected patterned light flux that has been guided to the ocular fundus Ef by the ring index projection optical system for measuring the ocular refractive power 43 and reflected by the ocular fundus Ef is condensed by the objective lens 41h. The condensed light flux is then reflected by the dichroic mirrors 41g, 41f and guided to the rotary prism 43i. In the light-receiving optical system 44, the reflected patterned light flux passes through the hole part 44a of the holed prism 43h via the rotary prism 43i. The reflected patterned light flux passed through the hole part 44a is then reflected by the mirror 44b and is formed into the reflected patterned light flux (i.e., the ring index for measuring the ocular refractive power) on the imaging element 44d (to be specific, on the light-receiving surface thereof) by the lens 44c. The imaging element 44d outputs image signals representing the formed image to the control unit 33. The control unit 33 displays an image of the ring index for measuring the ocular refractive power on the display 14 in accordance with the inputted image signals. As explained above, the light-receiving optical system 44 can form an image of the ring index for measuring the ocular refractive power on the light-receiving surface of the imaging element 44d and can display the image thereof on the display 14.

The alignment-light projection system 45 includes an LED 45a, a pinhole 45b, and a lens 45c, and shares the half mirror 42a with the observation optical system 42 and shares the dichroic mirror 41g and the objective lens 41h with the fixation target projection optical system 41. In the alignment-light optical system 45, a light flux emitted from the LED 45a passes through the pinhole 45b to be an alignment index light flux. The alignment index light flux is reflected by the half mirror 42a through the lens 45c and is guided to the main optical axis O10 of the ocular characteristics measurement unit 40. The alignment index light flux is guided to the objective lens 41h through the dichroic mirror 41g. The alignment index light flux is then projected onto the cornea Ec of the eye to be examined E as the alignment index light flux. With this, the alignment-light projection system 45 automatically aligns the main body 13 relatively to the eye E. Specifically, the alignment index light flux projected onto the eye E as a parallel beam is reflected by the cornea Ec of the eye E such that a bright point image is projected onto the imaging element 42d by the observation optical system 42 as an alignment index image. The alignment is completed by locating the projected bright point image within an alignment mark, which is formed or imaged by another optical system (not shown).

The ring index light sources for measuring the corneal shape 46A, 46B, and 46C are disposed at the front side of the objective lens 41h. Precisely, the light sources 46A, 46B, and 46C are disposed on a ring pattern 47 coaxially with the main optical axis O10 and at a position separated by a predetermined distance from the eye to be examined E (cornea Ec). The light sources 46A, 46B, and 46C project ring index light fluxes for measuring the corneal shape onto the eye to be examined E (cornea Ec). The ring index light fluxes for measuring the corneal shape are projected onto the cornea Ec of the eye E to form ring indexes for measuring the corneal shape 51 (shown in FIG. 7, etc.). The ring indexes 51 (specifically, light fluxes thereof) are reflected by the cornea Ec of the eye E and is formed into an image on the imaging element 42d by the observation optical system 42. Therefore, the observation optical system 42 can display the image of the ring indexes 51 together with the image of the anterior ocular segment (cornea Ec) on the display 14, as shown in FIG. 7.

The ocular characteristics measurement unit 40 has a driver (driving mechanism) to turn on/off the fixation target light source 41a, illumination light source of the observation optical system 42, light source for measuring the ocular refractive power 43a, LED 45a, and the ring index light sources for measuring the corneal shape 46A, 46B, and 46C. The driver is connected to the control unit 33. That is to say, the ocular characteristics measurement unit 40 is controlled by the control unit 33 to turn on/off the fixation target light source 41a, illumination light source of the observation optical system 42, light source 43a for measuring the ocular refractive power, LED 45a, and the ring index light sources for measuring the corneal shape 46A, 46B, and 46C. As explained above, the ocular characteristics measurement unit 40 is controlled by the control unit 33 to move the fixation target unit 41U integrally along the optical axis O11 by the fixation target moving mechanism 41D, to move the index unit 43U integrally along the optical axis O13 and the imagine element 44d along the optical axis O14 by the index moving mechanism 43D. The ocular characteristics measurement unit 40 is further controlled by the control unit 33 to generate an image based on the image signals, which are outputted from the imaging elements 42d and 44d, and to display the generated image on the display 14.

Next, overall operation to measure the shape of the cornea Ec of and the ocular refractive power (the spherical power, cylindrical power, cylinder axis angle, and the like) of the eye to be examined E using the ocular characteristics measurement unit 40 will be explained. The following operation of the ocular characteristics measurement unit 40 is executed by the control unit 33.

First, the examiner turns on the power switch of the ophthalmologic apparatus 10 and manipulates the display 14 (touch panel) to start a measurement using the ocular characteristics measurement unit 40. The ocular characteristics measurement unit 40 then sets the ocular characteristics measurement mode (shown in FIG. 5B; explained later), and illuminates the illumination light source of the observation optical system 42 to display an image of the anterior ocular segment (cornea Ec) on the display 14. The examiner then manipulates the input section of the display 14 to perform a rough alignment of the main body 13 relatively to the eye to be examined E by moving the main body 13 in the up-and-down direction and the left-and-right direction such that the pupil Ep of the eye E is displayed on the display 14. Further, the ocular characteristics measurement unit 40 (to be precise, the control unit 33) can detect the position and size (i.e., an area where the pupil Ep exists) of the pupil Ep from the image of the anterior ocular segment, which is formed or generated based on the image signals inputted from the imaging element 42d. For example, a shape to be recognized as a pupil Ep is pre-stored in a memory of the ocular characteristics measurement unit 40, and the ocular characteristics measurement unit 40 can detect the pupil Ep by recognizing the pre-stored shape based on contrasts of the generated image. The ocular characteristics measurement unit 40 may move the main body 13 in the left or right direction and detect the pupil Ep based on the image when changing the eye to be examined E between the left eye and right eye. With this, the ocular characteristics measurement unit 40 can use the detected position of the pupil as a target position of a rough alignment so as to move the main body 13 (the ocular characteristics measurement unit 40) to automatically perform the rough alignment.

The ocular characteristics measurement unit 40 displays the bright point image on the display 14 as the alignment index image using the observation optical system 42, and then commences alignment detection using the alignment-light projection system 45 and operation distance detection optical system (not shown). In other words, the ocular characteristics measurement unit 40 performs the automatic alignment by moving the main body 13 relatively to the base 11 in the up-and-down direction (Y-axis direction), the forward-and-backward direction (Z-axis direction), and the left-and-right direction (X-axis direction) such that the bright point image, which is used as the alignment index image, is located within the alignment mark. With this, the ocular characteristics measurement unit 40 completes the automatic alignment of the main body 13 relatively to the corneal apex Ea of the eye to be examined E. Note the ocular measurement unit 40 may turns on the ring index projection light sources for measuring the corneal shape 46A, 46B, and 46C when performing the automatic alignment so as to determine whether the measurement light (ocular refractive power measurement light) is properly projected onto the eye E (specifically, onto the anterior ocular segment, i.e., the cornea Ec).

The ocular characteristics measurement unit 40 then turns on the light source for measuring ocular refractive power 43a of the ring index projection optical system for measuring the ocular refractive power 43 to project a patterned light flux as the ring index for measuring the ocular refractive power onto the eye to be examined E, resulting in imaging of forming the ring index (patterned light flux) on the ocular fundus Ef through the pupil Ep of the eye E. The patterned light flux (ring index for measuring the ocular refractive power) is, as explained above, guided to the imaging element 44d of the light-receiving optical system 44. The control unit 33 then calculates the ocular refractive power (i.e., the spherical power, cylindrical power, cylinder axis angle, and the like) based on the image displayed on the display 13 (i.e., image signals outputted from the imaging element 44d) using a conventional technique. The ring index projection optical system 43 configures the ocular refractive power measurement optical system together with the light-receiving optical system 44, and also configures a measurement light projection optical system of the ocular characteristics measurement unit 40. Hence, the patterned light flux (i.e., the ring index for measuring the ocular refractive power) functions as measurement light for measuring the ocular refractive power (in this specification, it may also be called an ocular refractive power measurement light).

The ocular characteristics measurement unit 40 turns on the ring index projection light sources for measuring the corneal shape 46A, 46B, and 46C to project the ring index light fluxes for measuring the corneal shape onto the cornea Ec of the eye to be examined E. The reflected light fluxes from the anterior ocular segment (cornea Ec) of the eye E (i.e., the reflected light fluxes of the ring index light fluxes for measuring the corneal shape) are guided to the imaging element 42d of the observation optical system 42 as explained above. The control unit 33 then measures the corneal shape based on the image displayed on the display 13 (i.e., image signals outputted from the imaging element 42d). In other words, the control unit 33 measures the corneal shape of the eye E based on the image of the ring indexes for measuring the corneal shape 51 which are projected onto the cornea Ec (shown in FIG. 7, etc.). The details of how to measure the corneal shape is identical to that of a conventionally known technique, and thus is not explained herewith. The ring index light sources 46A, 46B, and 46C configure a corneal shape measurement optical system together with the observation optical system 42, and also configure a measurement light projection optical system of the ocular characteristics measurement unit 40. Hence, the ring index light fluxes for measuring the corneal shape function as measurement light for measuring the corneal shape (in this specification, it may also be called corneal shape measurement light).

Note the arrangement or the configuration of the ocular refractive power measurement unit (ocular characteristics measurement unit 40) is identical to the one disclosed in Japanese Patent Application No. 2002-253506 but should not be limited thereto. The control unit 33, as explained above, can execute both the measurement of the corneal shape and the measurement of the ocular refractive power (optical characteristics of the eye). Although not illustrated, the control unit 33 also has a memory to store the calculation results.

In the ophthalmologic apparatus 10 according to this embodiment, the intraocular pressure measurement unit 20 is typically disposed above the ocular characteristics measurement unit 40 of the main body 13, and the intraocular pressure measurement unit 20 and the ocular characteristics measurement unit 40 are fixed to a mounting base (not shown). In other words, the intraocular pressure measurement unit 20 and ocular characteristics measurement unit 40 are integrally configured such that the relative positions do not change. As a result, the mounting base and the intraocular pressure measurement unit 20 and ocular characteristics measurement unit 40 mounted thereon are moved by the driver 12 (the Y-axis driver 12a, Z-axis driver 12b, and X-axis driver 12c) relatively to the base 11 in the up-and-down direction (Y-axis direction), forward-and-backward direction (Z-axis direction), and left-and-right direction (X-axis direction). In other words, the main body 13 (mounting base) is moved by the driver 12 (Y-axis driver 12a, Z-axis driver 12b, and X-axis driver 12c) relatively to the base 11 such that the intraocular pressure measurement unit 20 and ocular characteristics measurement unit 40 are respectively moved to an appropriate position corresponding to the position of the eye to be examined (face of the subject) that is fixed by the chin rest 15 and forehead pad 16 (shown in FIGS. 5A and 5B).

Accordingly, the eye to be examined E is positioned on the extended line of the optical axis O1 of the anterior ocular segment observation optical system 21 of the intraocular pressure measurement unit 20 (i.e., the measurement unit 20 is moved to a position corresponding to the height of the eye E) by moving the main body 13 (mounting base) relatively to the base 11 in the up-and-down direction (Y-axis direction) with the Y-axis driver 12a (shown in FIG. 5A). Further, the tip of the air-puff nozzle 21b of the anterior ocular segment observation optical system 21 of the measurement unit 20 is moved to a position separated by a first predetermined distance (interval d1) from the eye E (specifically, from the ocular apex Ea) by moving the main body 13 in the forward-and-backward direction (Z-axis direction) with the Z-axis driver 12b. The interval d1 may be set to 11 mm, for example. This state illustrated in FIG. 5A is the intraocular pressure measurement mode.

Further, the eye to be examined E is positioned on the extended line of the main optical axis O10 of the ocular characteristics measurement unit 40 (i.e., the measurement unit 40 is moved to a position corresponding to the height of the eye E) by moving the main body 13 (mounting base) relatively to the base 11 in the up-and-down direction (Y-axis direction) with the Y-axis driver 12a (shown in FIG. 5B). The front end of the ocular characteristics measurement unit 40 is moved to a position separated by a second predetermined distance (interval d2) from the eye E (specifically, from the ocular apex Ea) by moving the main body 13 in the forward-and-backward direction (Z-axis direction) with the Z-axis driver 12b. Note that the front end of the measurement unit 40 means a component closest to the subject, i.e., the ring pattern 47 in this embodiment. The interval d2 may be set to about 80 mm, for example. This state illustrated in FIG. 5B is the intraocular pressure measurement mode.

The ophthalmologic apparatus 10 according to this embodiment displaces the front end of the intraocular pressure measurement unit 20 (i.e., air-puff nozzle 21b) to be on the positive side in the Z-axis direction (i.e., the subject side) more than the front end of the ocular characteristics measurement unit 40 (i.e., the ring pattern 47) due to a following reason. As explained and shown in FIG. 5A, the ring pattern 47 is positioned closest to the nose or mouth of the subject in the intraocular pressure measurement mode. Besides, the relative positions of the intraocular pressure measurement unit 20 and ocular characteristics measurement unit 40 do not change. Hence the ring pattern 47 may contact with nose or mouth of the subject or may give uncomfortable feeling to the subject even without any contact in the intraocular pressure measurement mode if the front ends of the intraocular pressure measurement unit 20 and the ocular characteristics measurement unit 40 are set to be the same position with respect to the Z-axis direction (forward-and-backward direction). The ophthalmologic apparatus 10 according to the embodiment, therefore, displaces the front end of the intraocular pressure measurement unit 20 (i.e., air-puff nozzle 21b) to be on the positive side in the Z-axis direction more than the front end of the ocular characteristics measurement unit 40 (i.e., ring pattern 47), thereby ensuring a space in front of nose or mouth of the subject in the intraocular pressure measurement mode.

The ocular characteristics measurement unit 40 of the ophthalmologic apparatus 10 according to this embodiment sets a reference interval (corresponding the interval d2) between the eye to be examined E (precisely, the corneal apex Ea) and the front end of the ocular characteristics measurement unit 40 (i.e., the ring pattern 47 in this embodiment) to be equal to or longer than 75 mm (e.g., about 80 mm in this embodiment) in the ocular characteristics measurement mode. The reference interval represents a standard position to where the front end of the ocular characteristics measurement unit 40 is moved relatively to the eye E when executing the ocular characteristics measurement.

A moving amount from the standard position to the positive side or negative side in the Z-axis direction is experimentally set in advance, e.g., ±20 mm. Further, the front end of the intraocular pressure measurement unit 20 (i.e., the air-puff nozzle 21b) is displaced to be on the positive side in the Z-axis direction more than the front end of the ocular characteristics measurement unit 40 (i.e., the ring pattern 47) as explained above. The air-puff nozzle 21b, which is the front end of the intraocular pressure measurement unit 20, is opposed to the forehead pad 16, which fixes the face of the subject together with the chin rest 15, in the ocular characteristics measurement mode as shown in FIG. 5B. Therefore, if the reference interval is too small, the front end of the intraocular pressure measurement unit 20 (i.e., the air-puff nozzle 21b) may contact with the forehead pad 16 when moving the ocular characteristics measurement unit 40 to the positive side in the Z-axis direction (i.e., to the subject's side) by the above moving amount. In other words, it is required to have a relatively large interval as the reference interval such that the ocular characteristics measurement unit 40 can move to the positive side in the Z-axis direction (i.e., to the subject's side) by the above moving amount. Consequently, the ocular characteristics measurement unit 40 of the ophthalmologic apparatus 10 according to this embodiment sets the reference interval (corresponding the interval d2) between the eye to be examined E (precisely, the corneal apex Ea) and the front end of the ocular characteristics measurement unit 40 (i.e., the ring pattern 47) to be equal to or longer than 75 mm, particularly about 80 mm in this embodiment.

Note that setting of the reference interval is adjusted by arranging optical characteristics of the optical members configuring the ocular characteristics measurement unit 40. Hence, the ophthalmologic apparatus 10 according to this embodiment can prevent the front end of the intraocular pressure measurement unit 20 (specifically, the air-puff nozzle 21b) from contacting with the forehead pad 16 in the ocular characteristics measurement mode although the front end of the intraocular pressure measurement unit 20 is displaced to be on the positive side in the Z-axis direction more than the front end of the ocular characteristics measurement unit 40 (specifically, the ring pattern 47).

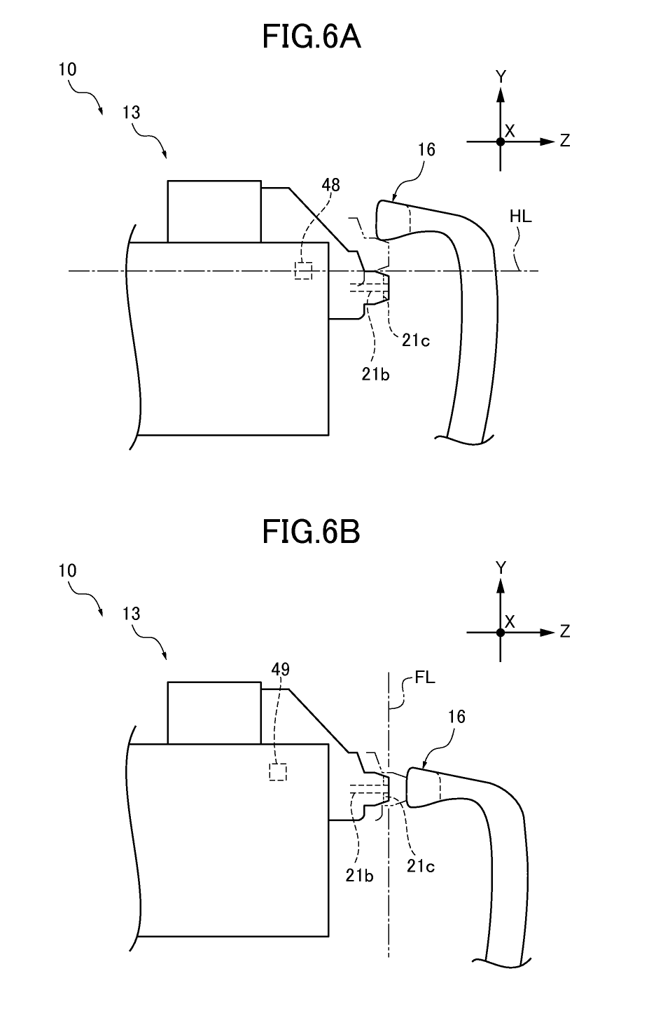

The ophthalmologic apparatus 10 according to this embodiment installs a height position detector 48 at the main body 13 to detect that the height of the air-puff nozzle 21b reaches to a predetermined height HL as shown in FIG. 6A. The predetermined height HL is set such that it can prevent the air-puff nozzle 21b of the intraocular pressure measurement unit 20 from contacting with the forehead pad 16. Note the predetermined height HL should appropriately be decided and should not be limited to the example as illustrated in FIG. 6A. The height position detector 48 outputs a signal to the control unit 33 when the height of the main body 13 (specifically, the air-puff nozzle 21b) reaches to the predetermined height HL. In a case where the control unit 33 receives the signal from the height position detector 48 and the intraocular pressure measurement mode has been selected, or in a case where the control unit 33 receives the signal and the intraocular pressure measurement unit 20 is positioned on the positive side in the Z-axis direction (on the subject's side) more than a prearranged forward-and-backward position; the control unit 33 stops or rejects the main body 13 to move upward. The prearranged forward-and-backward position is set such that it can prevent the air-puff nozzle 21b from contacting with the forehead pad 16 in the Z-axis direction.

The control unit 33 selects a control in accordance with a condition of the movement of the main body 13 after stopping the main body 13 to move upward. To be specific, the control unit 33 may display a warning on the display 14 to notify that the main body 13 is not able to move upward anymore if the control unit 33 receives the signal from the height position detector 48 when the main body 13 was being moved upward in response to the manipulation inputted to the display 14. Or, the control unit 33 may move the main body 13 backward (on the negative side in the Z-axis direction) and move the main body 13 upward if the control unit 33 receives the signal from the height position detector 48 when the main body 13 was being moved upward to change the mode from the intraocular pressure measurement mode to the ocular characteristics measurement mode. The control unit 33 may redo the detection of the eye to be examined E if the control unit 33 receives the signal from the height position detector 48 when the intraocular pressure measurement unit 20 was executing the automatic alignment. This is because that reaching to the predetermined height HL when the intraocular pressure measurement unit 20 is executing the automatic alignment means the automatic alignment fails to detect the eye E.

Upon turning on the power switch of the ophthalmologic apparatus 10 of the embodiment, the control unit 33 determines whether the signal from the height position detector 48 has been outputted before moving the main body 13. When it is determined that the signal is not outputted from the height position detector 48, the control unit 33 moves the main body 13 upward. On the other hand, when it is determined that the signal is outputted from the detector 48, the control unit 33 selects a control in accordance with a condition of the movement of the main body 13 without moving the main body 13 upward. To be specific, the control unit 33 may display a warning on the display 14 to notify that the main body 13 is not able to move upward anymore if the examiner manipulates the display 14 to move the main body 13 upward while the control unit 33 is receiving the signal from the detector 48. Or, the control unit 33 may move the main body 13 backward (on the negative side in the Z-axis direction) and move the main body 13 upward if the examiner commands to change the mode from the intraocular pressure measurement mode to the ocular characteristics measurement mode while the control unit 33 is receiving the signal from the detector 48. With this, even if the power switch is turned off during changing the measurement mode, it is possible to resume moving the main body 13 without contacting the air-puff nozzle 21b with the forehead pad 16 when the power switch is turned on again. Note that although the detector 48 of this embodiment is installed at in the main body 13 as shown in FIG. 6A, the position to install the height position detector 48 should not be limited thereto. For example, the detector 48 can be disposed nearby the Y-axis driver 12a, which is used to control the position in the Y-axis direction, or even at other places.

The ophthalmologic apparatus 10 according to this embodiment also installs a frontward position detector 49 at the main body 13 to detect that the frontward position of the air-puff nozzle 21b reaches to a predetermined frontward position FL as shown in FIG. 6B. The predetermined frontward position FL is set such that it can prevent the air-puff nozzle 21b from contacting with the forehead pad 16. For example, the predetermined frontward position is set such that the distance between the tip of the air-puff nozzle 21b and the forehead pad 16 has at least 1 mm. Note the predetermined frontward position FL should appropriately be decided and should not be limited to the example as illustrated in FIG. 6B. The frontward position detector 49 outputs a signal to the control unit 33 when the front end of the main body 13 (specifically, the tip of the air-puff nozzle 21b) reaches to the predetermined position FL. The control unit 33 stops or rejects the main body 13 to move forward when the control unit 33 receives the signal from the frontward position detector 49.

The control unit 33 selects a control in accordance with a condition of the movement of the main body 13 after stopping the main body 13 to move forward. To be specific, the control unit 33 may display a warning on the display 14 to notify that the main body 13 is not able to move forward (frontward) anymore if the control unit 33 receives the signal from the frontward position detector 49 when the main body 13 was being moved forward in response to the manipulation inputted to the display 14.

Upon turning on the power switch of the ophthalmologic apparatus 10 of the embodiment, the control unit 33 determines whether the signal from the frontward position detector 49 has been outputted before moving the main body 13. When it is determined that the signal is not outputted from the frontward position detector 49, the control unit 33 moves the main body 13 forward. On the other hand, when it is determined that the signal is outputted from the detector 49, the control unit 33 selects a control in accordance with a condition of the movement of the main body 13 without moving the main body 13 forward. To be specific, the control unit 33 may display a warning on the display 14 to notify that the main body 13 is not able to move forward (frontward) anymore if the examiner manipulates the display 14 to move the main body 13 forward while the control unit 33 is receiving the signal from the detector 49. Note that although the detector 49 of this embodiment is installed at the main body 13 as shown in FIG. 6B, the position to install the frontward position detector 49 should not be limited thereto. For example, the detector 49 can be disposed nearby the Z-axis driver 12b, which is used to control the position in the Z-axis direction, or even at other places.

The ophthalmologic apparatus 10 according to the embodiment normally measures the shape of the cornea Ec (corneal shape) and the ocular refractive power (spherical power, cylindrical power, and cylinder axis angle) of the eye to be examined E using the ocular characteristics measurement unit 40, and then measures the intraocular pressure of the eye E using the intraocular pressure measurement unit 20. As explained above, the control unit 33 controls the operation of each unit in response to the manipulations of the display 14 (i.e., commands inputted by the examiner via the display 14).

When the power switch of the ophthalmologic apparatus 10 according to the embodiment is turned on, the control unit 33 displays an intraocular pressure measurement icon for selecting the intraocular pressure measurement mode, and an ocular characteristics measurement icon for selecting the ocular characteristics measurement mode. Note that the display 14 may display one single icon for switching over the measurement mode, i.e., an icon for changing or selecting the modes, and should not be limited thereto.

The ophthalmologic apparatus 10 drives the Y-axis driving part 12a, Z-axis driving part 12b, and X-axis driving part 12c to change the mode into the ocular characteristics measurement mode (shown in FIG. 5B) when the examiner touches the ocular characteristics measurement icon on the display 14 to measure the corneal shape and the ocular refractive power (spherical power, cylindrical power, cylinder axis angle, and the like) of the eye E to be examined. Specifically, the apparatus 10 moves the driving parts 12a, 12b, and 12c such that the eye E is placed on the extended line of the main optical axis O10 of the ocular characteristics measurement unit 40. That is to say, the objective lens 41h of the fixation target projection optical system 41 of the ocular characteristics measurement unit 40 is opposed to the eye E. Further, the distance between the front end of the ocular characteristics measurement unit 40 and the eye E is set to the second predetermined distance (interval d2).

After setting the positions, the ocular characteristics measurement unit 40 measures the corneal shape and the ocular refractive power (spherical power, cylindrical power, cylinder axis angle, and the like) of the eye to be examined E, as explained above.

Following to the operation of the ocular characteristics measurement unit 40, the ophthalmologic apparatus 10 drives the Y-axis driving part 12a, Z-axis driving part 12b, and X-axis driving part 12c to change the mode into the intraocular pressure measurement mode (shown in FIG. 5A) when the examiner touches the intraocular pressure measurement icon on the display 14 to measure the intraocular pressure of the eye to be examined E. Specifically, the ophthalmologic apparatus 10 first moves the main body 13 to the negative side in the Y-axis direction until the height of the intraocular pressure measurement unit 20 reaches to that of the eye E. Additionally, the apparatus 10 controls the focusing mechanism 21D to move the CCD camera 21i such that the CCD camera 21i is positioned to the second focal position f2 on the optical axis O1. As a result, the ophthalmologic apparatus 10 can display an appropriate image of the eye E (specifically, the anterior ocular segment, i.e., cornea Ec) on the display 14.

The apparatus 10 then moves the main body 13 (intraocular pressure measurement unit 20) to the positive side in the Z-axis direction to close to the eye E until the distance between the tip of the air-puff nozzle 21b and the eye E becomes equal to the first predetermined distance (interval d1) as shown in FIG. 5A. Additionally, the apparatus 10 controls the focusing mechanism 21D to move the CCD camera 21i such that the CCD camera 21i is positioned to the first focal position f1 on the optical axis O1. As a result, the ophthalmologic apparatus 10 can display an appropriate image of the eye to be examined E (specifically, the anterior ocular segment, i.e., cornea Ec) on the display 14.

After setting the positions, the intraocular pressure measurement unit 20 measures the intraocular pressure of the eye E, as explained above.

Consequently, the ophthalmologic apparatus 10 according to the embodiment can measure the shape of the cornea Ec and the ocular refractive power (spherical power, cylindrical power, cylinder axis angle, and the like) of the eye to be examined E with the ocular characteristics measurement unit 40 and measures the intraocular pressure of the eye E with the intraocular pressure measurement unit 20.

A characteristic configuration of the ophthalmologic apparatus 10 according to the embodiment will be explained with reference to FIGS. 7-10. Note a measurement area Am (specifically, inside of a center-ring index image 51b of the ring indexes for measuring the corneal shape 51) is illustrated by hatching in the FIGS. 7-9 to easily understand the area Am.

As described above, the ring index light fluxes for measuring the corneal shape are projected from the ring index light sources for measuring the corneal shape 46A, 46B, and 46C onto the anterior ocular segment (i.e., cornea Ec) of the eye to be examined E to be formed into the ring indexes for measuring the corneal shape 51 (shown in FIG. 7, etc.) when the ophthalmologic apparatus 10 of the embodiment measures the ocular refractive power using the ocular characteristic measurement unit 40. The apparatus 10 also forms an image of the anterior ocular segment (cornea Ec) on the light-receiving surface of the imaging element 42d using the observation optical system 42 so as to obtain an image including the anterior ocular segment and the ring indexes 51, and outputs the image (data) to the control unit 33. As a result, the apparatus 10 can display the image obtained by the observation optical system 42 (to be specific, by the imaging element 42d) on the display 14 using the control unit 33, as shown in FIGS. 7-9.

In the ophthalmologic apparatus 10 according to the embodiment, the ring indexes for measuring the corneal shape 51 formed on the anterior ocular segment (cornea Ec) are three circles (specifically, an inner-ring index image 51a, the center-ring index image 41b, and an outer-ring index image 51c). These three circles are formed substantially concentric with each other as long as the anterior ocular segment (cornea Ec) of the eye E is in a normal condition. To be specific, the inner-ring index image 51a is formed by projecting the ring index light source for measuring the corneal shape 46A, the center-ring index image 51b is formed by projecting the ring index light source for measuring the corneal shape 46B, and the outer-ring index 51c is formed by projecting the ring index light source for measuring the corneal shape 46C.

The ophthalmologic apparatus 10 according to the embodiment sets the size of the center-ring image index 51b to be substantially as large as that of the measurement area Am. The measurement area Am represents an area where the ocular refractive power measurement light is projected onto the ocular fundus Ef by the ring index projection optical system for measuring the ocular refractive power 43 (the ocular refractive power measurement optical system) so as to measure the ocular refractive power as one of the ocular characteristics of the eye E. In other words, the measurement area Am indicates an area where the ring index projection optical system 43 (the ocular refractive power measurement optical system) can measure the ocular refractive power by using the ocular refractive power measurement light. The ocular refractive power measurement light is projected onto the ocular fundus Ef through the pupil Ep of the eye E. Therefore, it becomes possible to appropriately project the ocular refractive power measurement light onto the ocular fundus Ef of the eye E by completely locating the measurement area Am (to be specific, the center-ring index image 51b) within the pupil Ep, thereby measuring the ocular refractive power of the eye E properly.

Since the measurement area Am indicates the area where the ring index projection optical system for measuring the ocular refractive power 43 (the ocular refractive power measurement optical system) can measure the ocular refractive power by using the ocular refractive power measurement light, the center of the area Am coincides with the main optical axis O10. Further, since the ring indexes for measuring the corneal shape 51 are formed by the ring index light sources for measuring the corneal shape 46A, 46B, and 46C (corneal shape measurement optical system), the center of the ring indexes 51 also coincide with the main optical axis O10. Additionally, the inside of the center-ring index image 51b of the ring indexes 51 indicates the measurement area Am. In other words, the ophthalmologic apparatus 10 (precisely, the ocular characteristics measurement unit 40) according to the embodiment uses the ring index light fluxes for measuring the corneal shape of the eye to be examined E (measurement light), which is projected from the corneal shape measurement optical system, as measurement area index light fluxes (measurement area index light), which is used to indicate the measurement area Am on the anterior ocular segment of the eye E, to measure the ocular refractive power using the ocular refractive power measurement optical system (the ocular characteristics measurement unit 40). Accordingly, the ring index light sources 46A, 46B, and 46C (corneal shape measurement optical system) also function as a measurement area projection optical system, which projects measurement area index light fluxes on the main optical axis O10 of the ocular refractive power measurement optical system. Hence, the ring indexes 51 also function as the measurement area index light fluxes formed on the cornea Ec of the eye E.

As explained above, the control unit 33 of the ophthalmologic apparatus 10 according to the embodiment can detect the position and size of the pupil Ep of the eye to be examined E from the image (data) inputted from the imaging element 42d. For example, the shape to be recognized as the pupil Ep is pre-stored in the memory, and the control unit 33 can detect the pupil Ep by recognizing the pre-stored shape based on contrasts of the inputted image. Hence, the control unit 33 can detect an area indicating the pupil Ep from an anterior ocular segment.

The control unit 33 can detect the ring indexes for measuring the corneal shape 51 projected onto the cornea Ec of the eye E from the inputted image (data). For example, shapes to be recognized as the ring indexes 51 are pre-stored in the memory, and the control unit 33 can detect the shapes of the ring indexes 51 by recognizing the pre-stored shapes based on the contrasts of the imputed image. To be more specific, the control unit 33 compares the contrast values and/or brightness values with corresponding threshold values and can detect the shapes of the ring indexes 51 based on the results. With this, the control unit 33 can detect the ring indexes 51 (i.e., the ring index images 51a, 51b, and 51c) from the image of the anterior ocular segment.

As a result, the control unit 33 can detect the area surrounded by the center-ring index image 51b of the ring indexes 51. The area surrounded by the center-ring index image 51b represents the measurement area Am for the ocular refractive power measurement optical system of the ocular characteristics measurement unit 40, as explained above. That is to say, the control unit 33 can detect the measurement area Am by using the detected ring indexes 51.

Further, the control unit 33 can determine whether the detected ring indexes 51 (i.e., the inner-ring index image 51a, the center-ring index image 51b, and the outer-ring index image 51c) are properly projected onto the cornea Ec of the eye E. To be specific, each of the ring index images 51a, 51b, and 51c should be perfect circles and should not have a missing or breaking part if the ring indexes 51 are properly projected. In contrast, if the ring indexes 51 are improperly projected, for instance, if there is an obstacle in front of the anterior ocular segment (cornea Ec), the ring index images 51a, 51b, and 51c may have a missing or breaking part. The obstacle can be a closing eyelid or eyelashes shading the anterior ocular segment (cornea Ec). That is to say, since the ring indexes 51 are formed by projecting the ring index light fluxes from the ring index light sources 46A, 46B, and 46C, the projected light may be interrupted by the obstacle resulting in missing or breaking (so-called mechanical vignetting of) the ring indexes 51 (ring index images 51a-51c). The control unit 33 can determine that the detected ring indexes 51 are not in a proper condition for the measurement when a ratio of the area of the mechanical vignetting to the whole area of the detected ring index images 51a-51c exceeds a threshold value or when the number of breaking points on the circles (rings) exceeds a threshold value. When it is determined that the ring indexes 51 are not in the proper condition for the measurement, it also means that the ocular refractive power measurement light from the ocular refractive power measurement optical system is not properly projected onto the eye E either, i.e., the eye E is not in a good condition to measure the ocular refractive power. With this, the control unit 33 can determine whether the ring indexes for measuring the corneal shape 51 are in the proper condition for the measurement (i.e., whether the eye E is in a good condition to measure the ocular refractive power) by obtaining the shapes of the ring index images 51a-51c.

Note that as long as the control unit 33 can detect the pupil Ep and the ring indexes 51 (and their ring index images 51a-51c) from the image (data) inputted from the imaging element 42d, the way to detect them should not be limited to what explained above.

As explained, the control unit 33 can determine whether the measurement light (ocular refractive power measurement light) of the ocular characteristics measurement unit 40 is properly projected onto the anterior ocular segment (cornea Ec) of the eye to be examined E. Specifically, the control unit 33 determines that the measurement light is properly projected onto the anterior ocular segment (cornea Ec) of the eye E when the measurement area Am defined by the ring indexes for measuring the corneal shape 51 is appropriately located within the pupil Ep and the ring index images 51a-51c of the ring indexes 51 are properly projected onto the cornea Ec. As shown in FIG. 7, for example, the eyelid is fully open, the anterior ocular segment (cornea Ec) is not shaded by the eyelashes, and the ring indexes 51 (especially, the center-ring index image 51b) are appropriately located within the pupil Ep.

The control unit 33 determines that the measurement light is not properly projected onto the anterior ocular segment (cornea Ec) of the eye E when the ring index images 51a-51c are not in the proper condition for the measurement, as shown in FIG. 8. In this example shown in FIG. 8, the ring indexes 51 (the center-ring index image 51b) are appropriately located within the pupil Ep, but the anterior ocular segment (cornea Ec) is shaded by the eyelashes since the eyelid is not fully open.

The control unit 33 determines that the measurement light is not properly projected onto the anterior ocular segment (cornea Ec) of the eye E when the ring indexes 51 (especially, the center-ring index image 51b) are not appropriately located within the pupil Ep, as shown in FIG. 9. In this example shown in FIG. 9, the eyelid is fully open and the anterior ocular segment (cornea Ec) is not shaded by the eyelashes. However, the center-ring index image 51b is not appropriately located within the pupil Ep because the measurement light is not projected straight onto the eye E or because the center of the pupil Ep is not coincided with the center of the cornea Ec.

The control unit 33 stores the image (data), which is used for determining whether the measurement light is properly projected onto the anterior ocular segment (cornea Ec) of the eye E, into the memory. As explained, the image (data) is inputted from the imaging element 42d of the observation optical system 42.

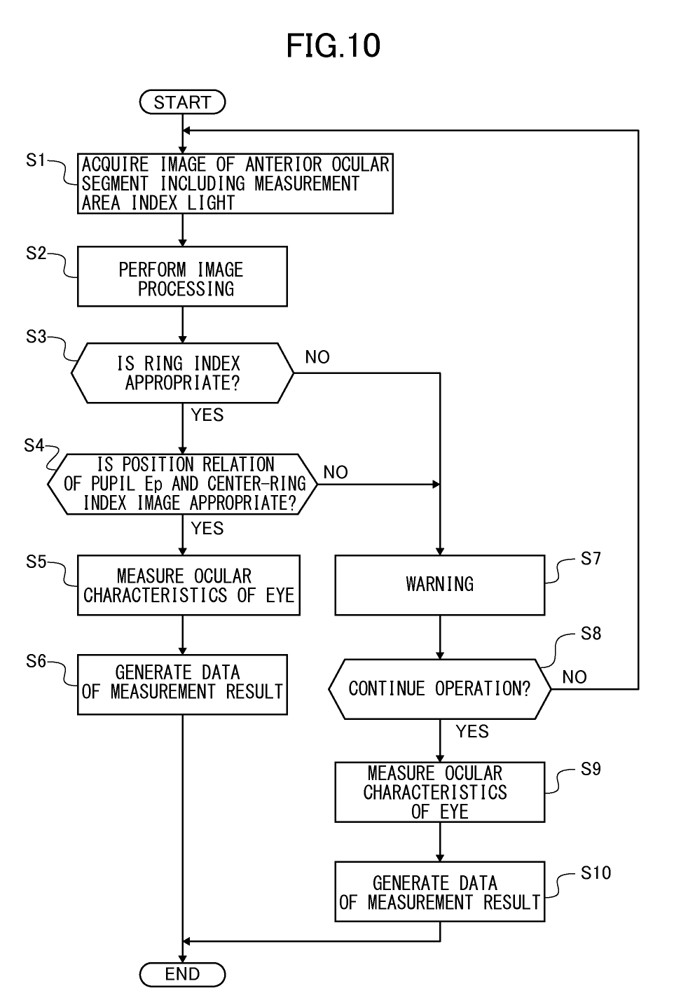

Here, an example of process to determine (or method for determining) whether the measurement light of the ocular characteristics measurement unit 40 is properly projected onto the anterior ocular segment (cornea Ec), which is executed by the control unit 33, will be explained with reference to FIG. 10. FIG. 10 is a flowchart showing the process (method) executed by the control unit 33 in accordance with a program installed in an internal (i.e., built-in) or external memory. Note that the following process (method) is commenced by performing the automatic alignment.

The program starts at Step S1, in which the control unit 33 acquires an image of the anterior ocular segment including a measurement area index light fluxes (ring index light fluxes for measuring the corneal shape). Specifically, in Step S1, the ocular characteristics measurement unit 40 turns on the ring index light sources for measuring the corneal shape 46A, 46B and 46C of the corneal shape measurement optical system, and the control unit 33 acquires the image of the anterior ocular segment from the light-receiving surface of the imaging element 42d of the observation optical system 42. With this, the ring index light fluxes are projected onto the cornea Ec as the measurement area index light fluxes to form the ring indexes for measuring the corneal shapes 51, and the control unit 33 can acquires the image (data) of the anterior ocular segment (cornea Ec) including the formed ring indexes 51. The control unit 33 also displays the image of the anterior ocular segment in accordance with the acquired image (data) on the display 14 (shown in FIG. 7). Note that the control unit 33 also performs the automatic alignment simultaneously.

Following to Step S1, the control unit 33 performs the image processing in Step S2. Specifically, in Step S2, the control unit 33 detects the pupil Ep (more specifically, the position and size of the pupil Ep) of the eye to be examined E from the processed image (data), detects the shapes of the ring indexes for measuring the corneal shape 51 (i.e., the ring index images 51a-51c) formed by the measurement area index light fluxes (ring index light fluxes for measuring the corneal shape), and detects the center-ring index image 51b and the measurement area Am, which is surrounded by the center-ring index image 51b.

Next, the program proceeds to Step S3, in which the control unit 33 determines whether the detected ring index images 51a-51c (ring indexes 51) are in the proper condition for the measurement. The program proceeds to Step S4 when the determination is affirmative, while the program proceeds to Step S7 when the determination is negative. As explained above, in Step S3, the control unit 33 determines whether the ring indexes 51 are in the proper condition based on the mechanical vignetting on the ring index images 51a-51c. In other words, in Step S3, the control unit 33 determines whether the eye E is in a good condition for the measurement of the ocular refractive power by using the ring indexes 51 (i.e., the ring index images 51a-51c) imaged on the image (data) acquired in Step S1. When it is determined that the ring indexes 51 are in the proper condition for the measurement, the program proceeds to Step S4 to determine whether the positional relationship of the pupil Ep and the center-ring index image 51b is appropriate. On the other hand, when it is determined that the ring indexes 51 are not in the proper condition for the measurement, the program proceeds to S7 and determines that the measurement light (ocular refractive power measurement light) is not properly projected onto the anterior ocular segment (cornea Ec) of the eye E. Note that the determination of Step S7 is made only if the ring indexes 51 have been inappropriate condition for the measurement even after repeating the automatic alignment.

Following to Step S3, when the determination in Step S4 is affirmative, i.e., when the control unit 33 determines that the positional relationship of the pupil Ep and the center-ring index image 51b is appropriate, the program proceeds to Step S5. On the other hand, when the determination in Step S4 is negative (and the ring indexes 51 have been in the improper condition even after repeating the automatic alignment), the program proceeds to Step S7. Specifically, in Step S4, the control unit 33 determines that the positional relationship of the pupil Ep and the center-ring index image 51b is appropriate when the center-ring index image 51b is appropriately located within the pupil Ep. On the other hand, the control unit 33 determines that the positional relationship is not appropriate when the center-ring index image 51b is not appropriately located within the pupil Ep. As explained above, since the center-ring index image 51b of the ring indexes 51 indicates the measurement area Am of the ring index projection optical system for measuring the ocular refractive power 43, the control unit 33 determines whether the measurement area Am is appropriately located within the pupil Ep based on the image including the anterior ocular segment and the center-ring index image 51b that are acquired in Step S1.

Following to Step S4, the control unit 33 measures the ocular characteristics of the eye to be examined E with the ocular characteristics measurement unit 40 in Step S5, and the program proceeds to Step S6. Particularly, the control unit 33 according to the embodiment measures the corneal shape and the ocular refractive power (optical characteristics) of the eye E in Step S5. Note the control unit 33 can concurrently determine whether the measurement light (ocular refractive power measurement light) is properly projected onto the anterior ocular segment (cornea Ec) of the eye E. This determination can be made by using the image (data) of the anterior ocular segment (cornea Ec) which includes the ring indexes 51 (i.e., the ring index images 51a-51c) obtained by the imaging element 42d of the observation optical system 42. On the other hand, the measurement of the ocular refractive power of the eye E can be made by using the image (data) of the patterned light flux which is reflected by the ocular fundus Ef and obtained by the imaging element 44d of the light-receiving optical system 44 (i.e., the other imaging element from the imaging element 42d of the observation optical system 42). As a result, the control unit 33 can executes the measurement and the determination concurrently.

Although not shown, when one imaging element functions as both the imaging element 42d of the observation optical system and the imaging element 44d of the ocular refractive power measurement optical system (ocular characteristics measurement unit 40), the control unit 33 can determine whether the measurement light is properly projected onto the eye E by using an image (data) of the anterior ocular segment (cornea Ec) that includes the ring indexes 51 and is taken immediately after the control unit 33 measures the ocular refractive power of the eye E with an image of the reflected patterned light flux. In this case, it is preferable to store the each image (data) used for the measurement and the determination by relating to each result.

Following to Step S5, the control unit 33 generates data regarding the measurement results of the ocular characteristics of the eye to be examined E in Step S6, and then finishes the process to determine (or method for determining) whether the measurement light is properly projected onto the anterior ocular segment (cornea Ec). Specifically, in Step S6, the control unit 33 stores the generated data into the internal or external memory. Note the control unit 33 may store the image (data), which is used for determining whether the measurement light (ocular refractive power measurement light) is properly projected onto the anterior ocular segment (cornea Ec) of the eye E, by relating to the generated data, and the control unit 33 may display the generated data onto the display 14 or output to the external device.

In Step S7, i.e., in a case where it is determined that the ring indexes for measuring the corneal shape 51 (the ring index images 51a-51c) are not in the proper condition in Step S3 or a case where it is determined that the positional relationship of the pupil Ep and the center-ring index image 51b is not appropriate in Step S4, the control unit 33 gives warning to the examiner, and the program proceeds to Step S8. Here, the warning is executed by, for example, outputting an alarm sound from an alarm outputting device installed at the main body 13. Instead, the warning may be executed by lighting or flashing a warning light installed at the main body 13, by displaying a warning message on the display 14, by vibrating a vibrator installed at the main body 13, or the like.

Following to Step S7, the control unit 33 determines whether the examiner continues the operation in Step S8. The program proceeds to Step S9 when the determination in Step S8 is affirmative, while the program returns to Step S1 when the determination in Step S8 is negative. Specifically in Step S8, the control unit 33 determines whether the examiner intends to perform the measurement even after the warning, i.e., even though the control unit 33 determines that the measurement light (ocular refractive power measurement light) of the ocular characteristics measurement unit 40 is not properly projected onto the anterior ocular segment (cornea Ec) of the eye to be examined E. For example, the examiner may intend or continue to perform the measurement when the examiner determines that there is no issue to perform the measurement. On the other hand, the examiner may stop the measurement when the eye E is not fully open. The determination of continuing the measurement in Step S8 may be made by using the display 14 with an icon for continuation of the measurement and an icon for checking the warning. The control unit 33 determines to continue the measurement and the program proceeds to Step S9 to execute the measurement of the ocular characteristics measurement unit 40 when the examiner touches the icon for continuation of the measurement. The control unit 33 determines not to continue the measurement and the program returns to Step S1 when the examiner touches the icon for checking the warning, such that the control unit 33 can determine or confirm whether the measurement light is properly projected onto the anterior ocular segment (cornea Ec) of the eye E by repeating the aforesaid process. Note that the control unit 33 may continue the measurement after a predetermined time period even if the examiner does not touch the icon for continuation.

Following to Step S8, the control unit 33 measures the ocular characteristics of the eye E with the ocular characteristics measurement unit 40, and the program proceeds to Step S10. Specifically, the control unit 33 measures the corneal shape and the ocular refractive power (optical characteristics) of the eye E with the measurement unit 40 in Step S9. Note the control unit 33 can concurrently determine whether the measurement light (ocular refractive power measurement light) of the ocular characteristics measurement unit 40 is properly projected onto the anterior ocular segment (cornea Ec) of the eye E in Step S9. Similar to Step S5, the control unit 33 can determine whether the measurement light of the ocular characteristics measurement unit 40 is properly projected onto the anterior ocular segment (cornea Ec) of the eye E by using the image (data) of the anterior ocular segment (cornea Ec) that includes the ring indexes 51 (i.e., the ring index images 51a-51c) and is taken immediately after measuring the ocular refractive power of the eye E in Step S9. In this case, it is preferable to store the image (data) used for the determination by relating to the measurement results, as explained regarding Step S5.

Following to Step S9, the control unit 33 generates data regarding the measurement results of the ocular characteristics of the eye to be examined E in Step S10, and finishes the process to determine (or method for determining) whether the measurement light is properly projected onto the anterior ocular segment (cornea Ec). Specifically, in Step S10, the control unit 33 stores the generated data into the internal or external memory. Note the control unit 33 may store the image (data), which is used for the determination whether the measurement light (ocular refractive power measurement light) is properly projected onto the anterior ocular segment (cornea Ec) of the eye E, by relating to the generated data. The control unit 33 may add color or hatching to a part corresponding to the inside of the center-ring index image 51b (i.e., a part corresponding to the measurement area Am) to the stored image (data), thereby facilitating to recognize the inside of the center-ring index image 51b of the ring indexes 51, i.e., the measurement area Am. The control unit 33 then displays the stored data with the related image (data) and information (e.g., an area size of the eyelid and/or the eyelashes covering the measurement area Am, an overlapping area size of the pupil Ep and the measurement area Am, and the like) on the display 14, or outputs them to the external device.

As mentioned above, the ophthalmologic apparatus 10 according to the embodiment of this invention includes the measurement area projection optical system (i.e., the ring index light sources for measuring the corneal shape 46A, 46B, and 46C) that projects measurement area index light fluxes (ring index light fluxes for measuring the corneal shape 51) onto the eye to be examined E. With this, the ophthalmologic apparatus 10 forms measurement area index light fluxes (i.e., the ring indexes 51) on the cornea Ec of the eye E. The apparatus 10 also includes the observation optical system 42 (specifically, the light-receiving surface of the imaging element 42d) that acquires an image of the eye E including the measurement area index light fluxes. The control unit 33 of the apparatus 10 then determines whether the measurement light (ocular refractive power measurement light) of the ocular characteristics measurement unit 40 is properly projected onto the eye E based on the image including the measurement area index light fluxes. To be specific, the control unit 33 can make the determination by detecting the position and the condition of the measurement area index light fluxes on the image of the eye E since the measurement area index light fluxes indicate the measurement area Am of the ocular refractive power measurement optical system on the anterior ocular segment of the eye E.

Further, the control unit 33 can determine that the measurement light is improperly projected onto the eye E and give a warning before executing the measurement. With this, it can avoid repeating the measurement. Besides, the control unit 33 can store the condition of the eye E at the time the measurement light is projected together with the index images (ring indexes for measuring the corneal shape 51). With this, it becomes possible to prevent from incorrectly determining an error of the intraocular optical system of the eye E regardless the measurement results.

The control unit 33 of the apparatus 10 can detect the position and size of the pupil Ep of the eye to be examined E from the image acquired by the observation optical system 42 (specifically, the light-receiving surface of the imaging element 42d). With this, it becomes possible to determine whether the measurement light is properly projected onto the eye E more accurately by using the detected position and the size of the pupil Ep together with the position and condition of the measurement area index light fluxes on the image.

In the ophthalmologic apparatus 10 according to the embodiment, the measurement area projection optical system also functions as the corneal shape measurement optical system (i.e., the ring index light sources 46A, 46B, and 46C and the observation optical system 42) that projects the corneal shape measurement light (i.e., the ring indexes for measuring the corneal shape 51) onto the eye E to measure the corneal shape. With this, it becomes possible to project the measurement area index light fluxes using the ring index light sources 46A, 46B, and 46C, which are originally installed and used to measure the corneal shape. Consequently, it becomes possible to achieve a simpler structure so as to apply the apparatus 10 according to the embodiment easily.

As explained, the apparatus 10 according to the embodiment of the invention can determine whether the measurement light (ocular refractive power measurement light) is properly projected onto the eye to be examined E.

In the abovementioned embodiment, the ocular characteristics measurement unit 40 and the intraocular pressure measurement unit 20 of this embodiment are integrally configured, and the ocular characteristics measurement unit 40 includes the ocular refractive power measurement optical system and corneal shape measurement optical system. However, this is only an example, and the configuration or the arrangement of the apparatus 10 should not be limited thereto. Any ocular characteristics measurement unit that projects measurement light onto the eye to be examined E (specifically, onto the ocular fundus Ef of the eye E), receives light reflected from the eye E, and measures the optical characteristics of the eye E (e.g., the ocular refractive power measurement optical system in the embodiment) can be applied to the apparatus 10.

In the abovementioned embodiment, the ring indexes for measuring the corneal shape 51, which include three ring index images (the ring index images 51a-51c) as corneal shape measurement light fluxes to measure the corneal shape of the eye to be examined E, are projected onto the eye E (to be specific, onto the anterior ocular segment (cornea Ec)). However, this is only an example and the corneal shape measurement light fluxes should not be limited thereto. For example, the corneal shape measurement light fluxes can be point group index light fluxes or include more or less ring index images.

In the abovementioned embodiment, the ophthalmologic apparatus 10 includes the ocular refractive power optical system as the ocular characteristics measurement unit. However, it should not be limited thereto. Any ocular characteristics measurement unit that receives light reflected by the ocular fundus Ef through the pupil Ep of the eye to be examined E so as to measure the optical characteristics of the eye E can be applied to the apparatus 10. For example, the ocular characteristics measurement unit may perform optical wave-front measurement, and/or have a different optical arrangement, different optical members with a different measuring theory, and the like.

In the abovementioned embodiment, the control unit 33 determines that the measurement light (ocular refractive power measurement light) is properly projected onto the eye E only when the measurement area Am is appropriately located within the pupil Ep as well as the ring indexes 51 (ring index images 51a-51c) are in the proper condition. However, the control unit 33 may determine that the measurement light is properly projected onto the eye E when the ring indexes 51 (ring index images 51a-51c) are in the proper condition regardless the positional relationship of the measurement area Am and the pupil Ep.

In the abovementioned embodiment, the apparatus 10 performs the automatic alignment. However, it should not be limited thereto, and this invention can be applied to an apparatus in which the examiner manually performs the alignment.

Although the present invention has been described in terms of exemplary embodiments, it is not limited thereto. It should be appreciated that variations may be made in the embodiments described by persons skilled in the art without departing from the scope of the present invention as defined by the following claims.

What is claimed is:

1. An ophthalmologic apparatus comprising:
an ocular characteristics measurement unit that measures an optical characteristic of an eye to be examined by projecting measurement light onto the eye and receiving light reflected by the eye;
an observation optical system that acquires an image of an anterior ocular segment of the eye;
a measurement area projection optical system that projects measurement area index light corresponding to a measurement area of the ocular characteristics measurement unit onto a cornea of the eye from a position defined in accordance with an optical axis of the ocular characteristics measurement unit; and
a control unit that controls the ocular characteristics measurement unit, the observation optical system, and the measurement area projection optical system;
wherein the control unit determines whether the measurement light is properly projected onto the eye based on the acquired image of the anterior ocular segment which includes the measurement area index light projected onto the cornea of the eye.

2. The apparatus as claimed in claim 1, wherein the control unit gives a warning when it is determined that the measurement light is improperly projected onto the eye.

3. The apparatus as claimed in claim 1, wherein the control unit performs alignment of the ocular characteristics measurement unit with respect to the eye when it is determined that the measurement light is improperly projected onto the eye, and the control unit determines whether the measurement light is properly projected onto the eye after performing the alignment.

4. The apparatus as claimed in claim 1, wherein the control unit starts a measurement performed by the ocular characteristics measurement unit when it is determined that the measurement light is properly projected onto the eye.

5. The apparatus as claimed in claim 1, wherein the control unit detects a position and size of a pupil of the anterior ocular segment from the acquired image of the anterior ocular segment.

6. The apparatus as claimed in claim 1, wherein the measurement area projection optical system functions as a corneal shape measurement optical system that projects corneal shape measurement light onto the cornea of the eye to measure a shape of the cornea.

7. The apparatus as claimed in claim 6, wherein the corneal shape measurement light is projected onto the cornea of the eye and is formed into a ring index for measuring the corneal shape on the eye.

8. The apparatus as claimed in claim 1, wherein the control unit stores a measurement result of the optical characteristic of the eye by relating to image data which is used to determine whether the measurement light is properly projected onto the eye.

9. The apparatus as claimed in claim 8, further including a display unit that is controlled by the control unit to display the acquired image of the anterior ocular segment and the measurement result,
wherein the control unit displays the stored measurement result and the stored image on the display unit.

10. The apparatus as claimed in claim 8, wherein the control unit outputs the stored measurement result and the stored image data to an external device.

11. A control method for an ophthalmologic apparatus that includes an ocular characteristics measurement unit, the method comprising:
a measuring step of measuring an optical characteristic of an eye to be examined by projecting measurement light onto the eye and receiving light reflected by the eye;
a projecting step of projecting measurement area index light corresponding to a measurement area of the ocular characteristics measurement unit onto a cornea of the eye;
an acquiring step of acquiring an image of an anterior ocular segment of the eye; and
a determining step of determining whether the measurement light is properly projected onto the eye based on the acquired image of the anterior ocular segment which includes the measurement area index light projected onto the cornea of the eye.

12. The method as claimed in claim 11, further including a warning step of giving a warning when it is determined that the measurement light is improperly projected onto the eye.

13. The method as claimed in claim 11, further including an alignment step of performing alignment of the ocular characteristics measurement unit with respect to the eye when it is determined that the measurement light is improperly projected onto the eye,
wherein the determining step determines whether the measurement light is properly projected onto the eye after the alignment step performs the alignment.

14. The method as claimed in claim 11, wherein the measuring step starts a measurement performed by the ocular measurement unit when it is determined that the measurement light is properly projected onto the eye.

15. The method as claimed in claim 11, further including a detecting step of detecting a position and size of a pupil of the anterior ocular segment based on the acquired image of the anterior ocular segment.
